SEQUENCE LISTING (16 sequences)

SEQ ID NO: 1 - protein, 132 aa, Homo sapiens
MKSSGLFPFLVLLALGTLAPWAVEGSGKSFKAGVCPPKKSAQCLRYKKPECQSDWQCPGKKRCCPDTCGIKCLDPVDTPNPTRRKPGKCPVTYGQCLMLNPPNFCEMDGQCKRDLKCCMGMCGKSCVSPVKA

SEQ ID NO: 2 - protein, 64 aa, Homo sapiens
MRVLYLLFSFLFIFLMPLPGVFGGIGDPVTCLKSGAICHPVFCPRRYKQIGTCGLPGTKCCKKP

SEQ ID NO: 3 - protein, 177 aa, Homo sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 4 - protein, 92 aa, Homo sapiens
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 5 - protein, 92 aa, Homo sapiens
MKLCVTVLSLLMLVAAFCSPALSAPMGSDPPTACCFSYTARKLPRNFVVDYYETSSLCSQPAVVFQTKRSKQVCADPSESWVQEYVYDLELN

SEQ ID NO: 6 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 7 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 8 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 9 - protein, 271 aa, Homo sapiens
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 10 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 11 - protein, 208 aa, Homo sapiens
MIIKHFFGTVLVLLASTTIFSLDLKLIIFQQRQVNQESLKLLNKLQTLSIQQCLPHRKNFLLPQKSLSPQQYQKGHTLAILHEMLQQIFSLFRANISLDGWEENHTEKFLIQLHQQLEYLEALMGLEAEKLSGTLGSDNLRLQVKMYFRRIHDYLENQDYSTCAWAIVQVEISRCLFFVFSLTEKLSKQGRPLNDMKQELTTEFRSPR

SEQ ID NO: 12 - protein, 67 aa, Homo sapiens
MRIHYLLFALLFLFLVPVPGHGGIINTLQKYYCRVRGGRCAVLSCLPKEEQIGKCSTRGRKCCRRKK

SEQ ID NO: 13 - DNA, 17 bp, Artificial Sequence
  primer
  misc_feature (9)..(9): n is a, c, g, or t
cctacgggnggcwgcag

SEQ ID NO: 14 - DNA, 21 bp, Artificial Sequence
  primer
gactachvgggtatctaatcc

SEQ ID NO: 15 - DNA, 16 bp, Artificial Sequence
  primer
agagtttgatcmtggc

SEQ ID NO: 16 - DNA, 20 bp, Artificial Sequence
  primer
ccgtcaattcmtttgagttt

(19) United States
(12) Patent Application Publication
(10) Pub. No.: US 2019/0339269 A1
(43) Pub. Date: Nov. 7, 2019

(54) COMPOSITIONS AND METHODS FOR PREDICTING RISK OF PRETERM BIRTH

(71) Applicants: The Trustees of the University of Pennsylvania, Philadelphia, PA (US); University of Maryland, Baltimore, Baltimore, MD (US)

(72) Inventors: Michal Elovitz, Wynnewood, PA (US); Jacques Ravel, Laurel, MD (US); Pawel Gajer, Takoma Park, MD (US)

(21) Appl. No.: 16/475,427

(22) PCT Filed: Jan. 3, 2018
(86) PCT No.: PCT/US2018/012185

§ 371 Date: Jul. 2, 2019

Related U.S. Application Data
(60) Provisional application No. 62/441,862, filed on Jan. 3, 2017.

(51) Int. Cl. G01N 33/569; C12Q 1/689; C12Q 1/6883

(52) U.S. Cl. CPC G01N 33/56911; G01N 33/56944; G01N 2800/368; C12Q 1/6883; C12Q 1/689

(57) ABSTRACT

Compositions, kits and methods for diagnosing and treating an increased risk of preterm birth (PTB) involves a composition comprising at least one reagent capable of detecting, binding, specifically complexing with, or measuring the level of one of a subject bacterium in a sample. Optionally, a composition of the invention further comprises a reagent capable of detecting, binding, specifically complexing with, or measuring the expression of a subject biomarker.

### STATEMENT OF GOVERNMENT INTEREST

This invention was made with government support under grant No. 5R01NR014784 awarded by the National Institute of Nursing Research. The government has certain rights in the invention.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “17-8103PCT_SEQ_LISTING”.

### BACKGROUND OF THE INVENTION

Preterm birth (PTB) is the leading cause of neonatal mortality and a significant contributor to neonatal morbidity. In the United States, approximately 12% of all live births are born preterm, i.e., before 37 weeks of gestational age. The extreme cost of PTB resides not only in the immediate neonatal care but also in long-term care of lasting morbidities resulting from prematurity. The care of preterm infants consumes a significant proportion of health care costs for children. Recent data suggest that there are more long-term sequelae from PTB than previously recognized including significant neurobehavioral abnormalities as these children reach school age. Effective prevention or treatment of PTB could significantly lower neonatal mortality and morbidity as well as health care costs. Therapies that target pregnant women at greatest risk for PTB are urgently needed. However, these types of interventions and therapeutic strategies cannot begin until we are able to reliably identify which women and infants are truly at greatest risk. To date, attempts at predicting which women will have PTB have not been successful. While some biomarkers have demonstrated high specificity, they lack sensitivity and positive predictive value making them a poor tool for risk stratification. It is of great clinical interest to identify biomarkers (either proteins in body fluids or in genetic ‘tendencies’) for the prediction of PTB allowing risk stratification, treatment and care to the highest risk group, while an identified ‘lower risk’ group can be spared unnecessary treatment, hospital stays, and concern of having a preterm neonate. Identifying those women at greatest risk for PTB will undoubtedly lead to new preventative strategies.

Although some progress has been made to date, there is still no reliable and definitive marker for preterm birth. There remains a need in the art for more accurate and sensitive diagnostic or predictive assays for PTB.

### SUMMARY OF THE INVENTION

In one aspect, a diagnostic composition or kit for predicting the risk of preterm birth is provided. The composition includes at least one reagent capable of detecting, binding, specifically complexing with, or measuring the level of one of a bacterium in a sample selected from:
a. Bifidobacterium breve;
b. Bifidobacterium longum;
c. Mobiluncus mulieris;
d. Arcanobacterium hippocoleae;
e. Streptococcus salivarius;
f. Peptoniphilus indolicus;
g. Gemella;
h. Eubacterium rectale;
i. BVAB2 (bacterial vaginosis associated bacteria 2)
j. BVAB3 (bacterial vaginosis associated bacteria 3)
k. Lactobacillus rhamnosus
l. Sneathia sanguinegens;
m. Lactobacillus gasseri;
n. g_Megasphaera (any species in genus)
o. BVAB1 (bacterial vaginosiseassociated bacteria 1)
p. Porphyromonas asaccharolytica
q. g Atopobium (any species in genus)
r. g_Prevotella (any species in genus, e.g., Prevotella buccalis, Prevotella genogroup 4)
s. Peptostrepococcus anaerobius
t. Gardnerella vaginalis
u. Lactobacillus crispatus; and
v. Lactobacillus iners.

In one embodiment, the bacteria are selected from: Mobiluncus mulieris, g Megasphaera (any species in genus), Sneathia sanguinegens, BVAB3, BVAB1, Porphyromonas asaccharolytica, g Atopobium (any species in genus), Prevotella buccalis, Peptostrepococcus anaerobius, Gardnerella vaginalis, Lactobacillus crispatus, and Lactobacillus iners. In one embodiment, the bacteria include Atopobium vaginae.

In one embodiment, the composition includes multiple reagents, each capable of detecting, binding, specifically complexing with, or measuring the level of one of a bacterium selected from (a)-(n). In one embodiment, the composition includes 3, 4, 5, 6, 7, 8, 9, 10, 11, 12, 13, 14, 15, 16, 17, 18, 19, 20, 21 or 22 of said reagents.

In another aspect, the composition includes a reagent capable of detecting, binding, specifically complexing with, or measuring the expression of a biomarker selected from:

i. Beta defensins;

ii. Secretory leukocyte inhibitory protease (SLPI);

iii. IL-1receptor antagonist (Ra);

iv. MIP1 alpha;

v. MIP1 beta;

vi. IL-1beta;

vii. IL-6;

viii. MCP-1;

ix. IL-1alpha;

x. Interferon-gamma; and

xi. Interferon-epsilon.

In one embodiment, the biomarkers are selected from beta-defensins, Interleukin-1receptor antagonist, Interleukin-1alpha, Interleukin-1beta, Interferon-gamma, Interferon-epsilon, and Secretory leukocyte inhibitory protease (SLPI).

In another aspect, a method of detecting the likelihood of occurrence of preterm birth is provided. The method includes detecting the presence or level of one or more bacterium in a patient sample, the bacterium selected from:
a. Bifidobacterium breve;
b. Bifidobacterium longum;
c. Mobiluncus mulieris;
d. Arcanobacterium hippocoleae;
e. Streptococcus salivarius;
f. Peptoniphilus indolicus;
g. Gemella;
h. Eubacterium rectale;
i. BVAB2;
j. BVAB3;
k. Lactobacillus rhamnosus;
l. Sneathia sanguinegens;
m. Lactobacillus gasseri;
n. g Megasphaera (any species in genus);
o. BVAB1;
p. Porphyromonas asaccharolytica;
q. g Atopobium (any species in genus);
r. g_Prevotella (any species in genus, e.g., Prevotella buccalis, Prevotella genogroup 4);
s. Peptostrepococcus anaerobius;
t. Gardnerella vaginalis;
u. Lactobacillus crispatus; and
v. Lactobacillus iners.

In one embodiment, the bacteria are selected from: Mobiluncus mulieris, g Megasphaera (any species in genus), Sneathia sanguinegens, BVAB3, BVAB1, Porphyromonas asaccharolytica, g_Atopobium (any species in genus), Prevotella buccalis, Peptostrepococcus anaerobius, Gardnerella vaginalis, Lactobacillus crispatus, and Lactobacillus iners. In one embodiment, the bacteria include Atopobium vaginae.

In another embodiment, the method further includes measuring the level of one or more of

i. Beta defensins;

ii. SLPI;

iii. IL-1Ra;

iv. MIP1 alpha;

v. MIP1beta;

vi. IL-1beta;

vii. IL-6;

viii. MCP-1;

ix. IL-1alpha;

x. Interferon-gamma; and

xi. Interferon-epsilon.

In one embodiment, the biomarkers are selected from Beta-defensins, Interleukin-1receptor antagonist, Interleukin-1alpha, Interleukin-1beta, Interferon-gamma, Interferon-epsilon, and Secretory leukocyte inhibitory protease (SLPI).

In a further aspect, a method of diagnosing and treating a subject for an increased risk of preterm birth is provided. In one embodiment, the method includes:

a. contacting a sample from the subject with a reagent capable of detecting, binding, specifically complexing with, or measuring the level of one of the bacterium from Table 1;

b. diagnosing the subject with an increased risk of preterm birth when the presence, increased or decreased relative abundance, or increased or decreased absolute abundance of a bacterium is detected; or the absence of low abundance of a bacterium;

c. administering an effective amount of a therapeutic to reduce the risk of PTB.

In another embodiment, the method includes:

a. contacting a sample from the subject with a reagent capable of detecting, binding, specifically complexing with, or measuring the level of at least one of the bacterium from Table 1;

b. contacting the sample with a reagent capable of detecting, binding, specifically complexing with, or measuring the level of at least one of the biomarkers from Table 2;

c. diagnosing the subject with an increased risk of preterm birth when the presence, increased or decreased relative abundance, or increased or decreased absolute abundance of a bacterium is detected, optionally in combination with a relatively low level of one or more of the biomarkers is present; and

d. administering an effective amount of a therapeutic to reduce the risk of PTB.

In another aspect, a kit is provided. In one embodiment, the kit includes reagents capable of detecting, binding, specifically complexing with or measuring the level of one or more of the bacteria from Table 1 and reagents capable of detecting, binding, specifically complexing with, or measuring the expression of one or more subject biomarkers.

Other aspects and advantages of the invention are described further in the following detailed description of the preferred embodiments thereof.

### BRIEF DESCRIPTION OF THE DRAWINGS

FIG. 1 is a graph of log 10 bacterial load v. gestation age in samples collected from women who delivered preterm and samples collected from women who delivered at term. The shaded regions are the 95% confidence intervals (CI).

FIGS. 2A-2E are 5 graphs showing the risk of spontaneous preterm birth (sPTB) as a function of the log relative abundance (x-axis) of the selected bacterial species.

FIG. 3 is a graph showing the odds ratio for sPTB associated with the presence of Mobiluncus mulieris and the listed immune biomarkers. The left bar shows the odds ratio for subjects having the listed immune biomarker level in the lowest 25% of the population and the right bar shows the odds ratio for subjects having the listed immune biomarker level in the highest 25% of the population. Samples were 83 cases of sPTB with 83 race-matched controls. Samples were taken at 16-20 weeks.

FIG. 4 is a graph showing the incidence of sPTB associated with the presence of Mobiluncus mulieris (MM) and Bifidobacterium breve (BB). From left to right, the bars show the incidence of sPTB with increasing levels of BB, from an absence of BB, to lowest 25%, to 25-50%, 50-57% and greater than 75% of the population. The bar on the far left shows the overall risk.

FIGS. 5A-5F are graphs showing the increasing rate of preterm birth (Y axis) with increasing load of bacteria (X-axis) for Sneathia sanguinegens (A), Mobiluncus curtisii and Mobiluncus mulieris (B), g_Megasphaera (C), BVAB3 (D), Porphyromonas asaccharolytica (E), and g_Atopobium (F).

FIG. 6 is a graph showing the −log₁₀ q-values of maximum change of risk of sPTB vs. maximum change of risk of sPTB. Relative abundance is shown for Sneathia sanguinegens, Mobiluncus curtisii mulieris, g_Megasphaera, BVAB1, Porphyromonas asaccharolytica, g_Atopobium and Prevotella buccalis from top to bottom. See Table 3.

FIG. 7 is a graph showing levels of cervicovaginal beta defensin by race and case/control status, for gestational weeks shown on the X axis. AA—African American.

FIG. 8 is a bar graph showing prevalence of sPTB by presence of select bacteria and human beta-defensin (BD) quartile. BD levels were measured in cervicovaginal fluid. For each set of bars grouped and noted on the y axis, each bar from left to right represents prevalence of sPTB by presence of BVAV3, MM, G. vag, BAVA1, or A. vag.

FIG. 9 is a “survival” curve by threshold for pregnant subjects. The bottom line represents subjects having a Mobiluncus mulieris level above a baseline threshold. Subjects having Mobiluncus mulieris levels of baseline threshold are shown in the top line. As pregnancy weeks progress (x-axis), the percentage of subjects still pregnant decrease. It can be seen that subjects with Mobiluncus mulieris levels above threshold drop off significantly, as PTL/PTB occurs.

FIG. 10 is a “survival” curve by threshold for pregnant subjects. The bottom line represents subjects having a BVAB3 level above a baseline threshold. Subjects having BVAB3 levels of baseline threshold are shown in the top line. As pregnancy weeks progress (x-axis), the percentage of subjects still pregnant decrease. It can be seen that subjects with BVAB3 levels above threshold drop off significantly, as PTL/PTB occurs.

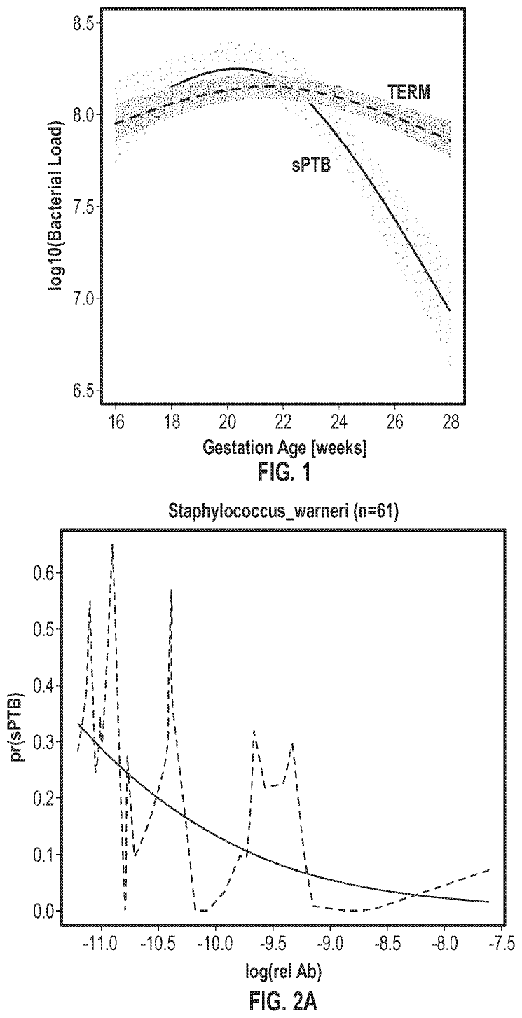

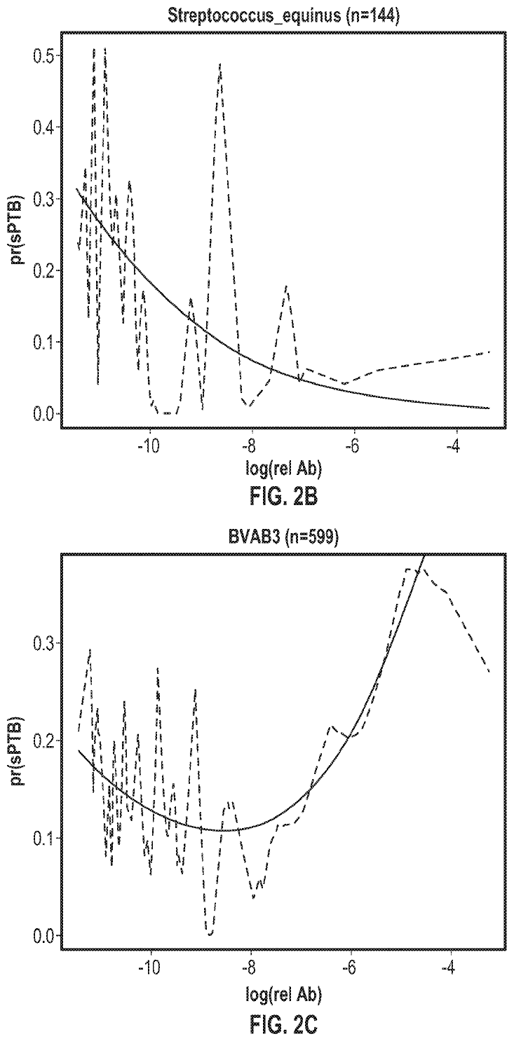

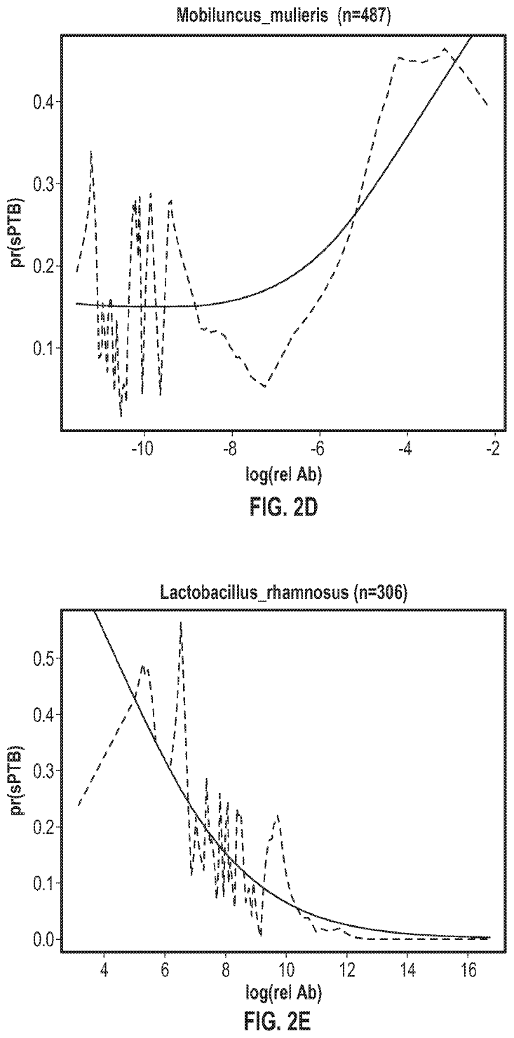

### DETAILED DESCRIPTION OF THE INVENTION

The present invention answers the need in the art by providing novel compositions and methods for diagnosing or predicting the likelihood of occurrence, or increased risk of, preterm birth utilizing the presence or level of one or more subject bacteria, as further described herein. In another aspect, the level of one or more subject biomarkers as described herein, is also, or alternatively, utilized in the diagnosis or risk assessment. The compositions and methods described herein, are suitable for use with both symptomatic and asymptomatic women.

“Increased likelihood” or “increased risk” of preterm birth, as used herein, means an increase in the risk or probability that the subject will develop preterm birth as compared to a predetermined control or baseline level. In one embodiment, increased likelihood means a 0.5 to 1 fold increase over the control or baseline level. In another embodiment, increased likelihood means a 1.0-1.5 fold increase over the control or baseline level. In one embodiment, increased likelihood means a 1.5 to 2 fold increase over the control or baseline level. In another embodiment, increased likelihood means a 2-3 fold increase over the control or baseline level. In another embodiment, increased likelihood means a 3-4 fold increase over the control or baseline level. In another embodiment, increased likelihood means a 4-5 fold increase over the control or baseline level. In another embodiment, increased likelihood means a 5-6 fold increase over the control or baseline level. In another embodiment, increased likelihood means a 6-7 fold increase over the control or baseline level. In another embodiment, increased likelihood means a 7-8 fold increase over the control or baseline level. In another embodiment, increased likelihood means an 8-9 fold increase over the control or baseline level. In another embodiment, increased likelihood means a 9-10 fold increase over the control or baseline level. In another embodiment, increased likelihood means a 10 fold or greater increase over the control or baseline level. Each of the numbers described above includes the endpoints and any fractions or integers therebetween. In one embodiment, the baseline risk of PTB is about 10%. However, this number may vary based on the subject population (e.g., race, socioeconomic status, smoking status, geographical location, etc.) In another embodiment, the baseline risk of PTB is about 11.2%, which has been reported as the baseline risk in the US. See, Ghartey et al, Am J Obstet Gynecol. 2015 June; 212(6): 776.e1-776.e12, which is incorporated herein by reference. In another embodiment, increased risk means a 10% greater risk over the control or baseline level. In another embodiment, increased risk means a 30% greater risk over the control or baseline level. In another embodiment, increased risk means a 30% greater risk over the control or baseline level. In another embodiment, increased risk means a 40% greater risk over the control or baseline level. In another embodiment, increased risk means a 50% greater risk over the control or baseline level. In another embodiment, increased risk means a 60% greater risk over the control or baseline level. In another embodiment, increased risk means a 70% greater risk over the control or baseline level. In another embodiment, increased risk means a 80% greater risk over the control or baseline level. In another embodiment, increased risk means a 90% greater risk over the control or baseline level. In another embodiment, increased risk means a 100% or greater risk over the control or baseline level.

“Decreased likelihood” or “decreased risk” of preterm birth, as used herein, means a decrease in the risk or probability that the subject will develop preterm birth as compared to a predetermined control or baseline level. In one embodiment, decreased likelihood means a 0.5 to 1.0 fold decrease over the control or baseline level. In another embodiment, decreased likelihood means a 1.0 to 1.5 fold decrease over the control or baseline level. In one embodiment, decreased likelihood means a 1.5 to 2 fold decrease over the control or baseline level. In another embodiment, decreased likelihood means a 2-3 fold decrease over the control or baseline level. In another embodiment, decreased likelihood means a 3-4 fold decrease over the control or baseline level. In another embodiment, decreased likelihood means a 4-5 fold decrease over the control or baseline level. In another embodiment, decreased likelihood means a 5-6 fold decrease over the control or baseline level. In another embodiment, decreased likelihood means a 6-7 fold decrease over the control or baseline level. In another embodiment, decreased likelihood means a 7-8 fold decrease over the control or baseline level. In another embodiment, decreased likelihood means an 8-9 fold decrease over the control or baseline level. In another embodiment, decreased likelihood means a 9-10 fold decrease over the control or baseline level. In another embodiment, decreased likelihood means a 10 fold or greater decrease over the control or baseline level. Each of the numbers described above includes the endpoints and any fractions or integers therebetween. In one embodiment, the baseline risk of PTB is about 10%. However, this number may vary based on the subject population (e.g., race, socioeconomic status, smoking status, geographical location, etc.) In another embodiment, decreased likelihood means about a 10% decrease over the control or baseline level. In another embodiment, decreased likelihood means about a 20% decrease over the control or baseline level. In another embodiment, decreased likelihood means about a 30% decrease over the control or baseline level. In another embodiment, decreased likelihood means about a 40% decrease over the control or baseline level. In another embodiment, decreased likelihood means about a 50% decrease over the control or baseline level. In another embodiment, decreased likelihood means about a 60% decrease over the control or baseline level. In another embodiment, decreased likelihood means about a 70% decrease over the control or baseline level. In another embodiment, decreased likelihood means about a 80% decrease over the control or baseline level. In another embodiment, decreased likelihood means about a 90% decrease over the control or baseline level. In another embodiment, decreased likelihood means about a 100% or greater decrease over the control or baseline level.

“Preterm birth” (PTB) as used herein means the birth of a baby at less than 37 weeks gestational age. In one embodiment, late preterm birth means birth of a baby between 34-37 weeks gestational age. In another embodiment, early preterm birth means birth of a baby at less than 34 weeks gestational age.

“Preterm labor” (PTL) as used herein, means the onset of labor symptoms at less than 37 weeks gestational age. Labor symptoms include cramps or contractions, watery discharge from the vagina, backache, severe pelvic pressure, and blood from the vagina. Preterm labor may or may not progress into preterm birth. “Diagnosis” as used herein, means determining, screening, or identifying the presence or level of expression of a bacterium or biomarker in a biological sample that indicates that a subject has an increased likelihood of developing a disease or condition.

“Diagnosis of PTB” as used herein means determining, screening, or identifying the presence or level of one or more subject bacteria, optionally, in combination with the level of expression of one or more subject biomarkers in a biological sample that indicates that a subject has an increased likelihood of developing PTB, or will develop PTB.

“Biological sample” or “sample” as used herein means any biological fluid or tissue that contains the subject bacteria or biomarkers. The most suitable sample for use in the methods described herein includes urine and cervicovaginal fluid (CVF). Other useful biological samples include, without limitation, whole blood, serum, plasma, urine, saliva, vaginal mucus, cervical mucus, placental fluid, saliva, placental cells or tissue, cells or tissue of the cervix, and cells or tissue of the vaginal wall. In some examples, “blood” may refer to any blood component used as a sample such as whole blood, plasma or serum. Such samples may further be diluted with saline, buffer or a physiologically acceptable diluent. Alternatively, such samples are concentrated by conventional means. In one embodiment, the sample is CVF. The sample may be obtained from the subject at any time of pregnancy. In one embodiment, the sample is obtained at the first clinical office visit, where pregnancy is verified. In another embodiment, the sample is obtained at 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37 weeks of pregnancy, including any days in between (e.g., 24 weeks, 3 days). In one embodiment, the sample is obtained at 16-20 weeks of pregnancy, also termed herein as V1. In one embodiment, the sample is obtained at 20-24 weeks of pregnancy, also termed herein as V2. In one embodiment, the sample is obtained at 24-28 weeks of pregnancy, also termed herein as V3. In another embodiment, the sample is obtained prior to the subject becoming pregnant. It is noted that in embodiments of the invention where both bacteria and biomarkers are detected, more than one sample may be used to detect the bacteria and biomarkers. For example, the bacteria may be detected in a sample of CVF while the biomarkers are detected in a sample of blood. Appropriate samples are readily selected by the person of skill in the art. In another embodiment, a single sample is used to detect the biomarkers and bacteria.

By the terms “patient” or “subject” as used herein is meant a female mammalian animal, including a human, a veterinary or farm animal, a domestic animal or pet, and animals normally used for clinical research, including non-human primates, dogs and mice. More specifically, the subject of these methods is a human. In one aspect of the methods described herein, the subject undergoing the diagnostic or therapeutic method is asymptomatic for preterm birth. In another aspect, the subject undergoing the diagnostic or therapeutic methods described herein shows clinical indicators, or history, of preterm birth.

“Clinical indicators of preterm birth” as used herein, include, but are not limited to, prior PTB, positive fetal fibronectin test, short cervical length, bacterial vaginosis, smoking, sexually transmitted diseases, African American race, low socio-economic status, stress, and depression. In one embodiment, the subject's race is used as a clinical indicator of PTB.

“Healthy subjects” or “healthy control” as used herein refer to a subject or population of multiple subjects that did not develop preterm birth. In one embodiment, healthy subjects may be a subject or population of multiple subjects that had preterm labor, but did not develop PTB. In another embodiment, healthy subjects may be a subject or population of multiple subjects that never developed PTL or PTB. In another embodiment, the healthy control is an artificial standard, such as that based on collected data from healthy subjects. Such artificial standard may be a standard such as that provided with a kit.

“Same time of pregnancy” as used herein, means that the sample was collected when the control subject was in the same gestational week of pregnancy as the test subject. In one example, the control subject may be one gestational week earlier or later than the test subject.

“Short cervix” or “short cervical length” (CL) as used herein means a cervical length, as measured by transvaginal ultrasound, of 25 mm or less. In one embodiment, a short CL has a measurement of 15 mm or less.

“Fetal fibronectin” (FFN) as used herein refers to the protein produced by fetal cells. FFN is found at the interface of the chorion and the decidua (between the fetal sack and the uterine lining). A positive FFN test refers to the presence of fetal fibronectin in the subject's vagina, i.e., in the cervicovaginal fluid. A positive fetal fibronectin (FFN) test is strongly associated with PTB while a negative test is a strong predictor of the pregnancy continuing for at least 14 more days.

As used herein, the “subject bacteria” refers to one or more of the bacterial species described herein, and contained in Table 1, below.

TABLE 1
Subject Bacteria
Subject bacteria

Bifidobacterium breve
Bifidobacterium longum
Prevotella genogroup 4
Mobiluncus mulieris
Arcanobacterium hippocoleae
Streptococcus salivarius
Peptoniphilus indolicus
Gemella
Eubacterium rectale
BVAB2
BVAB3
Lactobacillus rhamnosus
Sneathia sanguinegens
Lactobacillus gasseri
g_Megasphaera (any species in genus)
BVAB1
Porphyromonas asaccharolytica
Atopobium (any species in genus, e.g., Atopobium vaginae)
g_Prevotella (any species in genus, e.g., Prevotella buccalis)
Peptostrepococcus anaerobius
Gardnerella vaginalis
Lactobacillus crispatus
Lactobacillus iners

In one embodiment, the “subject bacteria” refers to one or more of:
a. Bifidobacterium breve;
b. Bifidobacterium longum;
c. Mobiluncus mulieris;
d. Arcanobacterium hippocoleae;
e. Streptococcus salivarius;
f. Peptoniphilus indolicus;
g. Gemella;
h. Eubacterium rectale;
i. BVAB2;
j. BVAB3;
k. Lactobacillus rhamnosus;
l. Snethia sanguinegens;
m. Lactobacillus gasseri;
n. g_Megasphaera (any species in genus);
o. BVAB1;
p. Porphyromonas asaccharolytica;
q. g Atopobium (any species in genus, e.g., Atopobioum vaginae);
r. g_Prevotella (any species in genus, e.g., Prevotella buccalis);
s. Peptostrepococcus anaerobius;
t. Gardnerella vaginalis;
u. Lactobacillus crispatus; and
v. Lactobacillus iners.

In one embodiment, the “subject bacteria” refers to one or more of Mobiluncus mulieris, g_Megasphaera (any species in genus), Sneathia sanguinegens, BVAB3, BVAB1, Porphyromonas asaccharolytica, g_Atopobium (any species in genus), Prevotella buccalis, Atopobium vaginae, Peptostrepococcus anaerobius, Gardnerella vaginalis, Lactobacillus crispatus, and Lactobacillus iners.

In one embodiment, the “subject bacteria” refers to one or more of Mobiluncus mulieris, BVAB3, Sneathia sanguinegens, g Atopobium (any species in genus, e.g., Atopobioum vaginae); and g_Prevotella (any species in genus, e.g., Prevotella buccalis).

In one embodiment, the “subject bacteria” includes Mobiluncus mulieris. In another embodiment, the subject bacteria includes BVAB3, In another embodiment, the subject bacteria includes Sneathia sanguinegens, In another embodiment, the subject bacteria includes g Atopobium (any species in genus, e.g., Atopobioum vaginae), In another embodiment, the subject bacteria includes g_Prevotella (any species in genus, e.g., Prevotella buccalis).

As used herein, the “subject biomarkers” refers to one or more of the biomarkers described herein, and contained in Table 2, below.

TABLE 2
Subject Biomarkers

beta defensins
SLPI
IL-1Ra
MIP1 alpha
IL-1b
IL-6
MIP1beta
MCP-1
IL-1a
Interferon gamma
Interferon epsilon

In one embodiment, the “subject biomarker” refers to one or more of beta defensin and SLPI, as further described below. In another embodiment, the “subject biomarker” refers to one or more of a beta defensin, SLPI, IL-1Ra, MIP1 alpha, MIP1 beta, IL-1a, IL-1b, IL-6, MCP-1, Interferon (IFN) gamma, and IFN epsilon. In another embodiment, the “subject biomarker” refers to one or more of beta-defensins, Interleukin-1receptor antagonist, Interleukin-1alpha, Interleukin-1beta, Interferon-gamma, Interferon-epsilon, and Secretory leukocyte inhibitory protease (SLPI). Any of isoforms of the subject biomarkers may be measured.

Secretory leukocyte protease inhibitor (SLPI), also known as antileukoproteinase, is an enzyme which modulates the inflammatory and immune responses after bacterial infection. Its inhibitory effect contributes to the immune response by protecting epithelial surfaces from attack by endogenous proteolytic enzymes. The amino acid sequence of SLPI is known in the art under, e.g., NCBI Reference Sequence: NP_003055.1 (SEQ ID NO: 1) and UniProtKB-P03973 (SLPI_HUMAN) while the nucleic acid coding sequence of SLIPI is known as, e.g., GenBank: AF114471.1 and nt 22-420 of NCBI Reference Sequence: NM_003064.3.

As used herein, beta defensin refers to any human beta defensin (HBD). The beta defensins are antimicrobial peptides implicated in the resistance of epithelial surfaces to microbial colonization. Defensins are 2-6 kDa, cationic, microbicidal peptides active against many Gram-negative and Gram-positive bacteria, fungi, and enveloped viruses, containing three pairs of intramolecular disulfide bonds. Such beta defensins include HBD-1, HBD2, HBD-3, HBD-5, HBD-6, HBD-7, HBD-8, HBD-9, HBD, 10, HBD-11, HBD-12, HBD-13, and HBD-14. See, Pazgier et al, Cellular and Molecular Life Sciences, 63(11):1294-1313 (June 2006). In one embodiment, the beta defensin is HBD-2. In another embodiment, the defensin is HBD-3. In another embodiment, “beta defensin” refers to one or more HBD.

Beta defensin 2 also known as skin-antimicrobial peptide 1 (SAP1) is a peptide that in humans is encoded by the DEFB4 (defensin, beta 4) gene. Human beta-defensin 2 is produced by a number of epithelial cells and exhibits potent antimicrobial activity against Gram-negative bacteria and Candida, but not Gram-positive S. aureus. It has been speculated that beta-defensin 2 may contribute to the infrequency of Gram-negative infections on skin and lung tissue. The amino acid sequence of beta defensin 2 is known in the art, e.g., NCBI Reference Sequence: NP_004933.1 (SEQ ID NO: 2), NP_001192195.1, UniProtKB-015263 (DFB4A_HUMAN) and GenBank: AAC33549.1, while the nucleic acid coding sequence of Beta defensin 2 is known as, e.g., nt 149-343 of NCBI Reference Sequence: NM_004942.3.

Beta defensin 3 (HBD-3) is a protein that in humans is encoded by the DEFB3 gene. HBD-3 was first isolated from human lesional psoriatic scales. RT-PCR showed HBD-3 to be expressed highly in skin, trachea, tongue and tonsils, with lower levels found salivary glands, uterus, kidney, bone marrow, thymus, colon, stomach, adenoid, pharynx, and larynx. The amino acid sequence of beta defensin 3 is known in the art, e.g., UniProtKB-P81534 (SEQ ID NO: 12, while the nucleic acid coding sequence of Beta defensin 2 is known as, e.g., nt 219-422 of NCBI Reference Sequence: NM_001081551.3.

The interleukin-1 receptor antagonist (IL-1RA or IL-1Ra) is a protein that in humans is encoded by the IL1RN gene. IL-1RA is a member of the interleukin 1 cytokine family. IL-1Ra is secreted by various types of cells including immune cells, epithelial cells, and adipocytes, and is a natural inhibitor of the pro-inflammatory effect of IL1β. Alternate isoforms are known in the art and include the sequences shown at Isoform 1: NP_776214.1 (SEQ ID NO: 3), Isoform 2: NP_776213.1, Isoform 3: NP_000568.1, and Isoform 4: NP_001305843. Each of these sequences is incorporated herein by reference. IL1RN gene and ranges of the coding DNA sequences (CDS) thereof is known in the art as, e.g., NCBI Reference Sequence: NM_173842.2 for transcript variant 1 with nt 65-598 as the CDS, NCBI Reference Sequence: NM_173841.2 for transcript variant 2 with nt 127-669 as the CDS, NCBI Reference Sequence: NM_000577.4 for transcript variant 3 with nt 127-606 as the CDS, NCBI Reference Sequence: NM_173843.2 for transcript variant 4 with nt 264-777 as the CDS, NCBI Reference Sequence: NM_001318914.1 for transcript variant 5 with nt 409-840 as the CDS, NCBI Reference Sequence: XM_011511121.1 for transcript variant X1 with nt 697-1128 as the CDS and NCBI Reference Sequence: XM_005263661.4 for transcript variant X2 with 194-625 as the CDS.

Macrophage Inflammatory Proteins (MIP) belong to the family of chemotactic cytokines known as chemokines. In humans, there are two major forms, MIP-1α and MIP-1β that are now officially named CCL3 and CCL4, respectively. Both are major factors produced by macrophages after they are stimulated with bacterial endotoxins. They are crucial for immune responses towards infection and inflammation. The amino acid sequence of MIP1 alpha is known in the art, and can be found, e.g., at NCBI Reference Sequence: NP_002974.1 (SEQ ID NO: 4) and as UniProtKB-P10147 (CCL3_HUMAN), while the nucleic acid coding sequence of MIP1 alpha is known as, e.g., nt 103-381 of NCBI Reference Sequence: NM_002983.2. The amino acid sequence of MIP1 beta is known in the art, and can be found, e.g., at NCBI Reference Sequence: NP_002975.1 (SEQ ID NO: 5) and as UniProtKB-P 13236 (CCL4_HUMAN), while the nucleic acid coding sequence of MIP1 beta is known as, e.g., nt 80-358 of NCBI Reference Sequence: NM_002984.3.

Interleukin 1 beta (IL1β) also known as “‘leukocytic pyrogen’”, “‘leukocytic endogenous mediator’”, “‘mononuclear cell factor’”, “‘lymphocyte activating factor’” and other names, is a cytokine protein that in humans is encoded by the IL1B gene, IL-1β is a member of the interleukin 1 family of cytokines. This cytokine is produced by activated macrophages as a proprotein, which is proteolytically processed to its active form by caspase. The amino acid sequence of IL-1beta is known in the art, and can be found, e.g., at, NCBI Reference Sequence: NP_000567.1 (SEQ ID NO: 6) and UniProtKB-P01584 (IL1B_HUMAN), while the nucleic acid coding sequence of IL-1beta is known as, e.g., nt 88-897 of NCBI Reference Sequence: NM_000576.2 and nt 182-898 of NCBI Reference Sequence: XM_017003988.1.

Interleukin 1 alpha (IL1a or IL1a or IL1alpha) also known as hematopoietin 1 is a cytokine of the interleukin 1 family that in humans is encoded by the IL1A gene. In general, Interleukin 1 is responsible for the production of inflammation, as well as the promotion of fever and sepsis. IL-1a inhibitors are being developed to interrupt those processes and treat diseases. IL-1a is produced mainly by activated macrophages, as well as neutrophils, epithelial cells, and endothelial cells. It possesses metabolic, physiological, haematopoietic activities, and plays one of the central roles in the regulation of the immune responses. It binds to the interleukin-1 receptor. It is on the pathway that activates tumor necrosis factor-alpha. The amino acid sequence of IL-1alpha is known in the art, and can be found, e.g., at UniProtKB/Swiss-Prot: P01583.1 (SEQ ID NO: 9), while the nucleic acid coding sequence of IL1beta is known as, e.g., nt 965-1780 of NCBI Reference Sequence: NM_000575.4.

Interleukin 6 (IL-6) is an interleukin that acts as both a pro-inflammatory cytokine and an anti-inflammatory myokine. In humans, it is encoded by the IL6 gene. Interleukin 6 is secreted by T cells and macrophages to stimulate immune response, e.g. during infection and after trauma, especially burns or other tissue damage leading to inflammation. IL-6 also plays a role in fighting infection, as IL-6 has been shown in mice to be required for resistance against bacterium Streptococcus pneumoniae. Alternate isoforms are known in the art and include the sequences shown at Isoform 1: NCBI Reference Sequence: NP_000591.1 (SEQ ID NO: 7) and Isoform 2: NCBI Reference Sequence: NP_001305024.1, while the nucleic acid sequence of IL6 is known as, e.g., NCBI Reference Sequence: NM_000600.4 for transcript variant 1 with nt 122-760 thereof as the CDS, NCBI Reference Sequence: NM_001318095.1 for transcript variant 2 with nt 159-569 thereof as the CDS, NCBI Reference Sequence: XM_005249745.4 for transcript variant X1 with nt 100-858 thereof as the CDS and NCBI Reference Sequence: XM_011515390.2 for transcript variant X2 with nt 455-1093 thereof as the CDS.

The chemokine (C-C motif) ligand 2 (CCL2) is also referred to as monocyte chemoattractant protein 1 (MCP1) and small inducible cytokine A2. CCL2 is a small cytokine that belongs to the CC chemokine family. CCL2 recruits monocytes, memory T cells, and dendritic cells to the sites of inflammation produced by either tissue injury or infection. The amino acid sequence of MCP-1 is known in the art, and can be found, e.g., at NCBI Reference Sequence: NP_002973.1 (SEQ ID NO: 8) and UniProtKB-P13500 (CCL2_HUMAN), while the nucleic acid sequence of MCP1 is known as, e.g., NCBI Reference Sequence: NM 002982.3 with nt 74-373 as the CDS.

Interferon gamma (IFNγ or IFNgamma) or type II interferon, is a cytokine that is critical for innate and adaptive immunity against viral, some bacterial and protozoal infections. IFNγ is an important activator of macrophages and inducer of Class II major histocompatibility complex (MHC) molecule expression. The amino acid sequence of IFNγ is known in the art, and can be found, e.g., at UniProtKB: P01579 (SEQ ID NO: 10), while the nucleic acid sequence of IFNgamma is known as, e.g., NCBI Reference Sequence: NM 000619.2 with nt 127-627 thereof as the CDS.

Interferon epsilon (IFNε) is a type I interferon, which is highly expressed in endometrial epithelial cells in proliferative phase of the menstrual cycle. IFNε is a potent anti-pathogen and immunoregulatory cytokine that may be important in combating STIs which represent a major global health and socioeconomic burden. The amino acid sequence of IFNε is known in the art, and can be found, e.g., at NCBI Reference Sequence: NP_795372.1 (SEQ ID NO: 11) and UniProtKB-Q86WN2 (IFNE_HUMAN), while the nucleic acid sequence of IFNε is known as, e.g., NCBI Reference Sequence: NM_176891.4 with nt 620-1246 thereof as the CDS.

As used herein, the term “predetermined control” refers to a numerical level, average, mean or average range of the expression of a biomarker in a defined population. The predetermined control level is preferably provided by using the same assay technique as is used for determination of presence or level of the subject bacteria. In another embodiment, predetermined control level is preferably provided by using the same assay technique as is used for determination of the measurement of the subject biomarker level(s). For example, the control may comprise a single healthy pregnant mammalian subject at the same time of pregnancy as the subject. In another embodiment, the control comprises a single healthy pregnant mammalian subject who did not develop preterm birth. In another embodiment, the control comprises a single healthy pregnant mammalian subject who had PTL, but did not develop PTB. In another embodiment, the control comprises a population of multiple healthy pregnant mammalian subjects at the same time of pregnancy as the subject or multiple healthy pregnant mammalian subjects who did not develop preterm birth. In another embodiment, the control comprises a population of multiple healthy pregnant mammalian subjects at the same time of pregnancy as the subject or multiple healthy pregnant mammalian subjects who had preterm labor but did not develop preterm birth. In another embodiment, the control comprises the same subject at an earlier time in the pregnancy. In another embodiment, the control comprises the same subject prior to pregnancy. In yet another embodiment, the control comprises one or multiple subjects with one or more clinical indicators of PTB, but who did not develop PTB. In addition, a predetermined control may also be a negative predetermined control. In one embodiment, a negative predetermined control comprises one or multiple subjects who had PTB.

As used herein, the term “control expression profile” refers to a numerical average, mean or average range of the expression of one or more subject biomarkers, or presence or level of one or more subject bacteria, in a defined population, rather than a single subject. For example, a positive control expression profile for one or more biomarker(s) in a healthy subject is a numerical value or range for expression of that biomarker(s) in a population of average healthy subjects who did not develop preterm birth. Likewise, a negative control expression profile for the expression of one or more biomarker(s) in a subject with preterm birth is a numerical value or range for the average expression of that biomarker(s) in a population composed of multiple patients who developed preterm birth.

Unless defined otherwise in this specification, technical and scientific terms used herein have the same meaning as commonly understood by one of ordinary skill in the art to which this invention belongs and by reference to published texts.

It should be understood that while various embodiments in the specification are presented using “comprising” language, under various circumstances, a related embodiment is also described using “consisting of” or “consisting essentially of” language. It is to be noted that the term “a” or “an”, refers to one or more, for example, “an immunoglobulin molecule,” is understood to represent one or more immunoglobulin molecules. As such, the terms “a” (or “an”), “one or more,” and “at least one” is used interchangeably herein.

Provided herein are compositions and methods which allow for the diagnosis, or prediction of the likelihood of occurrence of, preterm birth (PTB) or preterm labor (PTL). As used herein, when referring to a risk assessment, the terms PTB and PTL may be used interchangeably and refer to the onset of labor prior to 37 weeks of pregnancy, whether or not the baby is delivered prior to 37 weeks of pregnancy.

Provided herein are compositions and methods useful for diagnosing or predicting the likelihood of occurrence, or increased risk of, preterm birth utilizing the presence or level of one or more subject bacteria and/or one or more subject biomarkers, as further described herein.

A. Subject Bacteria

In part, the compositions and methods described herein relate to the presence of certain bacterial in the cervicovaginal space during pregnancy. It has been determined that the presence of certain bacteria have a protective, or risk-decreasing, effect on PTB, while others have a detrimental, or risk-increasing, effect on PTB.

In one embodiment, the composition or method includes reagents capable of checking for the presence of, or measuring the level of, a “subject bacteria” selected from those of Table 1.

In one aspect, a composition is provided which allows for the detection, or measurement of, one or more subject bacteria in a biological sample. In one embodiment, the composition includes at least one reagent capable of detecting, binding, specifically complexing with, or measuring the level of one of a bacterium in a sample selected from those in Table 1. In one embodiment, the composition includes multiple reagents, each capable of detecting, binding, specifically complexing with, or measuring the level of one of a bacterium selected from those of Table 1. In one embodiment, the composition includes 3, 4, 5, 6, 7, 8, 9, 10, 11, 12, 13, 14, 15, 16, 17, 18, 19, 20, 21 or 22 of said reagents. In another embodiment, a single set of reagents is provided which allows for detection or measurement of more than one bacteria selected from those of Table 1.

Reagents are provided herein which are capable of detecting, binding, specifically complexing with, or measuring the level of a subject bacterium. Such reagents are known in the art. In one embodiment, the reagents are those which are capable of detecting or measuring the amount or level of the bacterium at the nucleic acid level, e.g., via 16S rRNA or DNA. See, e.g., Janda and Abbott, 16S rRNA Gene Sequencing for Bacterial Identification in the Diagnostic Laboratory: Pluses, Perils, and Pitfalls, J. Clin. Microbiol. September 2007 vol. 45 no. 9 2761-2764, which is incorporated herein by reference. Kits for performing the same are available commercially, including, without limitation, MicroSEQ® 500 16S rDNA Bacterial Identification System (Applied Biosystems).

16S ribosomal rRNA gene sequencing and reagents therefor are useful in the compositions and methods described herein. 16S rRNAs are about 1500 nucleotides in length, and vary based on the bacterial species in question, particularly in hypervariable regions. For 16S rRNA gene sequencing it is possible to utilize universal primers which bind to conserved regions, and thus, are able to amplify 16S rRNA gene from various bacterial species. Such bacteria are then elucidated using sequencing and 16S rRNA gene sequence databases, such as, but not limited to, Bacterial 16S Ribosomal RNA RefSeq Targeted Loci Project found at ncbi.nlm.nih.gov/refseq/targetedloci. Thus, in one embodiment, 16S rRNA gene sequencing is used. Primers useful in 16S rRNA sequencing are known in the art, and may be designed by the person of skill in the art. See, e.g., Klindworth et al, Nucleic Acids Res. 2013 January; 41(1): e1, which is incorporated herein by reference. In one embodiment, the primer pairs are 5′-CCTACGGGNGGCWGCAG-3′ (SEQ ID NO: 13), and 5′-GACTACHVGGGTATCTAATCC-3 (SEQ IS NO: 14). In another embodiment, the primers are 5′-AGAGTTTGATCMTGGC-3′ (SEQ IS NO: 15), and S-D-Bact-0907-a-A-20, 5′-CCGTCAATTCMTTTGAGTTT-3′ (SEQ IS NO: 16). See Klindworth et al. Other suitable primers may be designed by the person of skill in the art.

In another embodiment, the diagnostic reagent is a polynucleotide or genomic probe that hybridizes to the bacterial DNA or RNA. Such polynucleotides may be about 20, about 22, about 25 or more nucleotides in length. In another embodiment, the diagnostic reagent is a PCR primer-probe set that amplifies and detects a polynucleotide sequence of the subject bacteria. In one embodiment, the reagent is immobilized on a substrate. In another embodiment, the diagnostic reagent comprises a microarray, a microfluidics card, a computer-readable chip or chamber.

In one embodiment, a diagnostic reagent for the subject bacteria is capable of detecting, binding, specifically complexing with the 16S rRNA of the subject bacteria. In a further embodiment, a diagnostic reagent of a subject bacteria comprises a polynucleotide, genomic probe, or a pair of PCR primers that hybridizes to the 16S rRNA of the subject bacteria. Suitable assays utilizing the described polynucleotide, genomic probe, or a pair of PCR primers may include but are not limited to PCR, quantitative PCR, southern blot analysis, dot-blot hybridization and fluorescence in situ hybridization (FISH).

Conventional methods or tools can be utilized by one of skill in the art in designing suitable polynucleotide, genomic probe, or a pair of PCR primers as described, in view of the subject bacteria disclosed herein, for example directed to the hypervariable region of the 16S rRNA. Examples of such tools include arb-silva.de/fish-probes/probe-design/, exiqon.com/custom-fish and decipher.cee.wisc.edu/DesignProbes.html. The 16S rRNA sequence of a subject bacteria can also be accessed by one of skill in the art from a publicly or commercially available database, e.g., rnacentral.org/. Exemplary sequences are provided herein. However, other sequences are known in the art. For example, a sequence identified as RNAcentral identifier URS0000884084_None is provided for Sneathia sanguinegens 16S ribosomal RNA, a sequence identified as RNAcentral identifier URS0000AE9A4C_1685 is provided for Bifidobacterium breve 16S ribosomal RNA, a sequence identified as RNAcentral identifier URS00004973ED_216816 is provided for Bifidobacterium longum 16S ribosomal RNA; a sequence identified as RNAcentral identifier URS0000B886DB_2052 is provided for Mobiluncus mulieris 16S ribosomal RNA; a sequence identified as RNAcentral identifier URS00009E1704_149017 is provided for Arcanobacterium hippocoleae partial 16S ribosomal RNA; a sequence identified as RNAcentral identifier URS000022965F_1304 is provided for Streptococcus salivarius 16S ribosomal RNA; a sequence identified as RNAcentral identifier URS000022D6DF_997350 is provided for Peptoniphilus indolicus 16S ribosomal RNA; a sequence identified as RNAcentral identifier URS00008A7D4F_657318 is provided for Eubacterium rectale 16S ribosomal RNA; a sequence identified as RNAcentral identifier URS00004D4F4F_47715 is provided for Lactobacillus rhamnosus 16S ribosomal RNA; a sequence identified as RNAcentral identifier URS00002492CC_1596 is provided for Lactobacillus gasseri 16S ribosomal RNA; a sequence identified as RNAcentral identifier URS000087DFC7_879243 is provided for Porphyromonas asaccharolytica 16S ribosomal RNA; a sequence identified as RNAcentral identifier URS000083D847_82135 is provided for Atopobioum vaginae 16S ribosomal RNA; a sequence identified as RNAcentral identifier URS00007CB734_1401074 is provided for Prevotella buccalis 16S ribosomal RNA; a sequence identified as RNAcentral identifier URS0000310CEA_698958 is provided for Gardnerella vaginalis 16S ribosomal RNA; a sequence identified as RNAcentral identifier URS0000A15CD5_47770 is provided for Lactobacillus crispatus 16S ribosomal RNA; and a sequence identified as RNAcentral identifier URS00002392DB_879299 is provided for Lactobacillus iners 16S ribosomal RNA are available to one of skill in the art.

Examples of the polynucleotide, genomic probe, or a pair of PCR primers for detecting, binding, specifically complexing with, or measuring the level of BVAB3 (also known as Bacterial Vaginosis-Associated Bacteria-3) can also be found in the following references which are incorporated herein by reference in their entireties: Marcela Zozaya-Hinchliffe et al, Quantitative PCR Assessments of Bacterial Species in Women with and without Bacterial Vaginosis. J Clin Microbiol. 2010 May; 48(5): 1812-1819. Published online 2010 Mar. 19; David N. Fredricks et al, Changes in Vaginal Bacterial Concentrations with Intravaginal Metronidazole Therapy for Bacterial Vaginosis as Assessed by Quantitative PCR, J Clin Microbiol, 2009 March; 47(3): 721-726, Published online 2009 Jan. 14; Ai Wen et al, Selected Vaginal Bacteria and Risk of Preterm Birth: An Ecological Perspective, The Journal of Infectious Diseases, Volume 209, Issue 7, 1 Apr. 2014, Pages 1087-1094 and the supplemental data. Published: 22 Nov. 2013; David N. Fredricks et al, Molecular Identification of Bacteria Associated with Bacterial Vaginosis. N Engl J Med. 2005 Nov. 3; 353(18):1899-911 and supplementary Appendix; and David N. Fredricks et al, Targeted PCR for Detection of Vaginal Bacteria Associated with Bacterial Vaginosis J Clin Microbiol. 2007 October; 45(10):3270-6. Epub 2007 Aug. 8.

Examples of the polynucleotide, genomic probe, or a pair of PCR primers for detecting, binding, specifically complexing with, or measuring the level of Mobiluncus mulieris can be found in the following references which are incorporated herein by reference in their entireties: David N. Fredricks et al, Molecular Identification of Bacteria Associated with Bacterial Vaginosis. N Engl J Med. 2005 Nov. 3; 353(18):1899-911 and supplementary Appendix; and Marcela Zozaya-Hinchliffe et al, Quantitative PCR Assessments of Bacterial Species in Women with and without Bacterial Vaginosis. J Clin Microbiol. 2010 May; 48(5): 1812-1819. Published online 2010 Mar. 19.Marcela Zozaya-Hinchliffe et al as cited above also provide an example of the polynucleotide, genomic probe, or a pair of PCR primers for detecting, binding, specifically complexing with, or measuring the level of Atopobium vaginae, Gardnerella vaginalis, BVAB1, B VAB2, B VAB3, Lactobacillus gasseri, Sneathia sanguinegens, Lactobacillus crispatus; and Lactobacillus iners.

Liselotte Hardy et al (Unravelling the Bacterial Vaginosis-Associated Biofilm: A Multiplex Gardnerella vaginalis and Atopobium vaginae Fluorescence In Situ Hybridization Assay Using Peptide Nucleic Acid Probes, PLoS One. 2015; 10(8): e0136658. Published online 2015 Aug. 25. doi: 10.1371/journal.pone.0136658) provide an example of the polynucleotide, genomic probe, or a pair of PCR primers for detecting, binding, specifically complexing with, or measuring the level of Atopobium vaginae and Gardnerella vaginalis.

Yukiko Miyamoto et al (Design of cluster-specific 16S rDNA oligonucleotide probes to identify bacteria of the Bacteroides subgroup harbored in human feces, FEMS Microbiology Letters, Volume 177, Issue 1, 1 Aug. 1999, Pages 143-149, doi.org/10.1111/j.1574-6968.1999.tb13725.x Published: 1 Aug. 1999) provide an example of the polynucleotide, genomic probe, or a pair of PCR primers for detecting, binding, specifically complexing with, or measuring the level of g_Prevotella.

Other conventional method of detecting, binding, specifically complexing with, or measuring the level of a subject bacteria and reagents utilized therein might be used in the compositions, methods, and kits described herein. For example, nitrocellulose filter blots can be used to identifying Mobiluncus mulieris, see, e.g., M C Roberts et al, Nitrocellulose filter blots for species identification of Mobiluncus curtisii and Mobiluncus mulieris. J Clin Microbiol. 1984 October; 20(4): 826-827. Other conventional tests include Gram stain and colony morphology, pigmentation, disk susceptibility to antimicrobials (kanamycin; vancomycin; colistin), spot indole test with paradimethyl-aminocinnamaldehyde, catalase reaction with hydrogen peroxide, sensitivity to bile on bile-esculin agar and Rapid ID 32A system. Where appropriate, further tests include the lipase reaction on egg yolk agar, indole production and nitrate reduction in indole-nitrate broth, pigmentation on laked rabbit blood agar, stimulation of growth by formate and fumarate or pyruvate, and motility from broth culture. Enzyme reactions for α-fucosidase, β-N-acetylghicosaminidase, β-xylosidase, α-glucosidase, trypsin, esculin hydrolysis, β-galactosidase (ONPG) and urease van be determined with Rosco tablets. Carbohydrate-fermentation tests may be performed in prereduced anaerobically sterilized (PRAS) media for glucose, arabinose, cellobiose, lactose, salicin, sucrose and xylose. Short-chain fatty acid analysis by gas-liquid chromatography (GLC) was performed on the PRAS peptone-yeast-glucose broth culture. See, e.g., Julie Downes et al, Evaluation of the Rapid ID 32A system for identification of anaerobic Gram-negative bacilli, excluding the Bacteroides fragilis group, Clin Microbiol Infect. 1999 June; 5(6):319-326.

In one embodiment, the composition includes reagents capable of detecting, binding, specifically complexing with, or measuring the level of one or more of Mobiluncus mulieris and Bifidobacterium breve. In another embodiment, the composition includes reagents capable of detecting, binding, specifically complexing with, or measuring the level of one or more of Bifidobacterium breve, Bifidobacterium longum, Prevotella genogroup 4, Mobiluncus mulieris, Arcanobacterium hippocoleae, Streptococcus salivarius, Peptoniphilus indolicus, Gemella and Eubacterium rectale. In another embodiment, the composition includes reagents capable of detecting, binding, specifically complexing with, or measuring the level of one or more of Mobiluncus mulieris, Bifidobacterium breve, Staphylococcus warneri, Streptococcus equinus, BVAB3, and Lactobacillus rhamnosus. In another embodiment, the composition includes reagents capable of detecting, binding, specifically complexing with, or measuring the level of one or more of Mobiluncus mulieris, g_Megasphaera (any species in genus), Sneathia sanguinegens, BVAB3, BVAB1, Porphyromonas asaccharolytica, g_Atopobium (any species in genus), Prevotella buccalis, Atopobium vaginae, Peptostrepococcus anaerobius, Gardnerella vaginalis, Lactobacillus crispatus, and Lactobacillus iners. In another embodiment, the composition includes reagents capable of detecting, binding, specifically complexing with, or measuring the level of one or more of Mobiluncus mulieris, Sneathia sanguinegens, g_Megasphaera, g_Atopobium (any species in genus), BVAB3, Porphyromonas asaccharolytica, Atopobium vaginae, and Prevotella buccalis. In another embodiment, the composition includes reagents capable of detecting, binding, specifically complexing with, or measuring the level of each of the bacteria of Table 1.

In another embodiment, the composition includes reagents capable of detecting, binding, specifically complexing with, or measuring the level of Mobiluncus mulieris and one or more other bacterium selected from those in Table I. In another embodiment, the composition includes reagents capable of detecting, binding, specifically complexing with, or measuring the level of Mobiluncus mulieris, BVAB3 and one or more other bacterium selected from those in Table I.

As discussed above, in certain embodiments, the subject bacteria include any bacteria from the Prevotella genus (g_Prevotella). Such species include, without limitation, Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, and Prevotella veroralis. In one embodiment, the Prevotella species is Prevotella buccalis.

As discussed above, in certain embodiments, the subject bacteria include any bacteria from the Megasphaera genus (g_Megasphaera). Such species include, without limitation, Megasphaera hominis, Megasphaera mincronuciformis, Megasphaera elsdenii, Megasphaera cerevisiae, Megasphaera paucivorans, and Megasphaera sueciensis.

As discussed above, in certain embodiments, the subject bacteria include any bacteria from the Atopobium genus (g_Atopobium). Such species include, without limitation, Atopobium deltae, Atopobium fossor, Atopobium minutum, Atopobium parvulum, Atopobium rimae, and Atopobium vaginae.

As discussed in the Examples below, certain of the subject bacteria have a detrimental effect on the risk of preterm birth. That is, the presence, relative abundance, or absolute abundance of these “detrimental bacteria” is indicative of an increased risk of preterm birth. Conversely, certain of the subject bacteria have a beneficial effect on the risk of preterm birth. That is, the presence, relative abundance, or absolute abundance of these “beneficial bacteria” is indicative of a decreased risk of preterm birth.

As used herein, the term “absolute abundance” relates to the total number of the specific bacterial strain in the biological sample. The term “relative abundance”, relates to the total number of the specific bacterial strain (or genus) in the biological sample, as compared to the total number of bacteria (the “total bacterial load”) in the sample. In one embodiment, the amount of total bacteria in the sample is measured as a function of the total 16s rRNA in the sample. See, e.g., Hopkins et al, Gut, 2001; 48:198-205, which is incorporated herein by reference.

In one embodiment, an increased risk of preterm birth is found when the absolute abundance of one or more subject bacteria is increased as compared to a control level. In another embodiment, an increased risk of preterm birth is diagnosed when the relative abundance of one or more subject bacteria is increased as compared to a control level. In yet another embodiment, an increased risk of preterm birth is diagnosed when the presence of one or more subject bacteria is detected in the sample. Such bacteria are termed detrimental bacteria.

In another embodiment, a decreased risk of preterm birth is diagnosed when the absolute abundance of one or more subject bacteria is increased as compared to a control level. In yet another embodiment, a decreased risk of preterm birth is diagnosed when the relative abundance of one or more subject bacteria is increased as compared to a control level. In another embodiment, a decreased risk of preterm birth is diagnosed when the presence of one or more subject bacteria is detected in the sample. Such bacteria are termed beneficial bacteria.

In one embodiment, a decrease in the total bacterial load of the sample is indicative of an increased risk of preterm birth.

In one embodiment, Staphylococcus warneri is a beneficial bacterium. In one embodiment, an increase in the absolute abundance or relative abundance of Staphylococcus warneri is indicative of a decrease in the risk of preterm birth as compared to a control. Conversely, a decrease in the absolute abundance or relative abundance of Staphylococcus warneri is indicative of an increase in the risk of preterm birth as compared to a control.

In one embodiment, Streptococcus equinus is a beneficial bacterium. In one embodiment, an increase in the absolute abundance or relative abundance of Streptococcus equinus is indicative of a decrease in the risk of preterm birth as compared to a control. Conversely, a decrease in the absolute abundance or relative abundance of Streptococcus equinus is indicative of an increase in the risk of preterm birth as compared to a control.

In one embodiment, Lactobacillus rhamnosus is a beneficial bacterium. In one embodiment, an increase in the absolute abundance or relative abundance of Lactobacillus rhamnosus is indicative of a decrease in the risk of preterm birth as compared to a control. Conversely, a decrease in the absolute abundance or relative abundance of Lactobacillus rhamnosus is indicative of an increase in the risk of preterm birth as compared to a control.

In one embodiment, BVAB3 is a detrimental bacterium. In one embodiment, an increase in the absolute abundance or relative abundance of BVAB3 is indicative of an increase in the risk of preterm birth as compared to a control. Conversely, a decrease in the absolute abundance or relative abundance of BVAB3 is indicative of a decrease in the risk of preterm birth as compared to a control.

In one embodiment, Sneathia sanguinegens is a detrimental bacterium. In one embodiment, an increase in the absolute abundance or relative abundance of Sneathia sanguinegens is indicative of an increase in the risk of preterm birth as compared to a control. Conversely, a decrease in the absolute abundance or relative abundance of Sneathia sanguinegens is indicative of a decrease in the risk of preterm birth as compared to a control.

In one embodiment, Mobiluncus mulieris is a detrimental bacterium. In one embodiment, an increase in the absolute abundance or relative abundance of Mobiluncus mulieris is indicative of an increase in the risk of preterm birth as compared to a control. Conversely, a decrease in the absolute abundance or relative abundance of Mobiluncus mulieris is indicative of a decrease in the risk of preterm birth as compared to a control.

In one embodiment, g_Megasphaera is a detrimental bacterium. In one embodiment, an increase in the absolute abundance or relative abundance of g_Megasphaera is indicative of an increase in the risk of preterm birth as compared to a control. Conversely, a decrease in the absolute abundance or relative abundance of g_Megasphaera is indicative of a decrease in the risk of preterm birth as compared to a control.

In one embodiment, Porphyromonas asaccharolytica is a detrimental bacterium. In one embodiment, an increase in the absolute abundance or relative abundance of Porphyromonas asaccharolytica is indicative of an increase in the risk of preterm birth as compared to a control. Conversely, a decrease in the absolute abundance or relative abundance of Porphyromonas asaccharolytica is indicative of a decrease in the risk of preterm birth as compared to a control.

In one embodiment, g_Atopobium is a detrimental bacterium. In one embodiment, an increase in the absolute abundance or relative abundance of g_Atopobium is indicative of an increase in the risk of preterm birth as compared to a control. Conversely, a decrease in the absolute abundance or relative abundance of g_Atopobium is indicative of a decrease in the risk of preterm birth as compared to a control.

In one embodiment, when Mobiluncus mulieris is present in the sample, a low SLPI level as compared to a control is indicative of an increased risk of PTB. In another embodiment, a SLPI level in the lowest 25% of a control population is indicative of about 3 times greater risk of PTB.

In one embodiment, when Mobiluncus mulieris is present in the sample, a low IL-1Ra level as compared to a control is indicative of an increased risk of PTB. In one embodiment, a IL-1ra level in the lowest 25% of a control population is indicative of about 2 times greater risk of PTB.

In another embodiment, when Mobiluncus mulieris is present in the sample, a low level of one or more or combined beta defensin(s) as compared to a control is indicative of an increased risk of PTB. In one embodiment, when Mobiluncus mulieris is present in the sample, a low level of one or more or combined beta defensin(s) level and/or IL1Ra as compared to a control is indicative of an increased risk of PTB. In one embodiment, a low level of one or more or combined beta defensin(s) and/or IL-1Ra level in the lowest 25% of a control population is indicative of about 5 times greater risk of PTB.

In another embodiment, when Atopobium vaginae is present in the sample, a low B-defensin level and/or IL1Ra as compared to a control is indicative of an increased risk of PTB. In one embodiment, a B-defensin level and/or IL-1Ra level in the lowest 25% of a control population is indicative of about 7 times greater risk of PTB. In another embodiment, a Beta defensins level in the lowest 25% of a control population is indicative of about 9 times greater risk of PTB.

In one embodiment, an increase in the absolute abundance or relative abundance of Mobiluncus mulieris and/or BVAB3 is indicative of an about 2 times or greater risk of preterm birth compared to control. In another embodiment, in the presence of Moliluncus mulieris in the sample, the absence of Bifidobacterium breve, or a Bifidobacterium breve level in the lowest 25% of a control population, is indicative of increased risk of PTB. In yet another embodiment, in the presence of Mobiluncus mulieris in the sample, a Bifidobacterium breve level in the highest 50% or 75% of a control population is indicative of decreased risk of PTB.

By “level in the lowest 25% of a control population” it is meant that, when a statistically significant sample population is measured, the subject's measured level falls within the bottom 25% of that level, or range of levels. Similarly, by “level in the highest 50% or 75% of a control population” it is meant that, when a statistically significant sample population is measured, the subject's measured level falls within the top 50% or 75% of that level, or range of levels. It is intended that a standard level may be determined based on sample population measurements, which may be substituted for a control population.

The control population may be selected by the person of skill in the art. In one embodiment, the control sample may be obtained from a healthy pregnant mammal at the same time of pregnancy as the subject. In another embodiment the control sample may be obtained from a healthy pregnant mammal who did not develop preterm birth. In another embodiment, the control sample may be obtained from a healthy pregnant mammal who had PTL, but did not develop PTB. In a further embodiment, the control sample may be obtained come from a population of multiple healthy subjects described above. In another embodiment, the control sample may be obtained from the same subject at an earlier time in the pregnancy. In another embodiment of the invention, the level of expression of the biological sample or predetermined control is a mean or average, a numerical mean or range of numerical means, a numerical pattern, a graphical pattern or an expression profile. In another embodiment, the control is a baseline level, such as when the risk of PTB is being determined. In another embodiment, the control sample may be obtained from the same subject prior to pregnancy.

It is contemplated that, in one embodiment of the compositions and methods herein, reagents are provided which allow for detection and/or measurement of one or more subject bacteria via PCR, while one or more subject biomarkers are detected and/or measured via ELISA.

### II. Subject Biomarkers

In another aspect, provided herein is a composition which includes a reagent capable of detecting, binding, specifically complexing with, or measuring the expression of a “subject biomarker” selected from those of Table 2. In one embodiment, the biomarkers are selected from beta-defensins, Interleukin-1 receptor antagonist, Interleukin-1alpha, Interleukin-1beta, Interferon-gamma, Interferon-epsilon, and Secretory leukocyte inhibitory protease (SLPI).

In one embodiment, the composition includes a reagent capable of detecting, binding, specifically complexing with, or measuring the expression of a biomarker selected from those of Table 2. In another embodiment, the composition includes multiple reagents, each capable of detecting, binding, specifically complexing with, or measuring the expression of a biomarker selected from those of Table 2. In one embodiment, the composition includes 3, 4, 5, 6, 7, 8, 9 10 or 11 of said reagents. In some embodiments, the composition includes reagents for the detection of subject bacteria and reagents for the detection of subject biomarkers.

In one embodiment, the composition includes a reagent capable of detecting, binding, specifically complexing with, or measuring the expression of a beta defensin. In one embodiment, the beta defensin is HBD2. In one embodiment, the beta defensin is HBD3. In another embodiment, the composition includes a reagent capable of detecting, binding, specifically complexing with, or measuring the expression of SLPI. In yet another embodiment, the composition includes a reagent capable of detecting, binding, specifically complexing with, or measuring the expression of IL-1Ra. In yet another embodiment, the composition includes reagents capable of detecting, binding, specifically complexing with, or measuring the expression of beta defensin, SLPI and/or IL-1Ra. It is intended that the compositions and methods described herein encompass reagents capable of detecting any combination of the subject biomarkers.

In one embodiment, the reagents are those which are capable of detecting, binding, specifically complexing with, or measuring the expression of a subject biomarker at the polypeptide or protein level. Such reagents are known in the art. In one embodiment, the reagent is an antibody or fragment thereof. In one embodiment, the antibody specifically binds to at least part of, i.e., a fragment or epitope of, the subject biomarker. Such fragments or epitopes include 8-15 amino acids, up to 25aa, up to 50aa, up to 75aa, up to 100aa, up to 150aa, up to 200aa, up to 300aa, up to 400aa, up to 500aa, up to 600aa, up to 700aa, up to 750aa. Such antibodies are known in the art, or may be developed.

Such reagents are useful in assays known to the person of skill in the art, such as an ELISA. Non-limiting examples of antibodies to the subject biomarkers are provided. However, it is noted that the sequence of each of the subject biomarkers are known and provided for convenience in the sequence listing. The person of skill in the art would readily be able to generate suitable reagents, e.g., antibodies, using known techniques. See, e.g., Greenfield, E. A., Chapter 7: Generating Monoclonal Antibodies in Antibodies: a Laboratory Manual, Second Ed., 2014.

Examples of antibodies to the subject biomarkers are as follows:

TABLE 3
Exemplary antibodies

| Subject Biomarkers | Exemplary Antibody | Source |
|---|---|---|
| HBD1 | ab14421 | Abcam |
| HBD2 | ab63982 | Abcam |
| HBD3 | ab19270 | Abcam |
| HBD4 | AF2758 | R&D Systems |
| SLPI | ab17157 | Abcam |
| IL-1Ra | ab124962 | Abcam |
| MIP1 alpha | CR3M | Invitrogen |
| IL-1b | MABF18 | Millipore Sigma |
| IL-6 | ab9324 | Abcam |
| MIP1beta | MAB451 | R&D Systems |
| MCP-1 | 2D8 | Invitrogen |
| IL-1a | ab46028 | Abcam |
| Interferon gamma | MAB2851 | R&D Systems |
| Interferon epsilon | 3B8 | Novus Biologicals |

Alternatively, such reagents are those that measure expression at the mRNA level. Such reagents can be readily designed using the DNA sequences of the biomarkers of Table II, which are known in the art, and noted herein. Suitable methods for mRNA isolation and quantification are known and can be used in conjunction with the nucleic acid sequences known in the art. Suitable systems include the PAXgene Blood RNA system (for collection and isolation of the mRNA) in conjunction with quantitative PCR.

In one embodiment, an increase in the level of one of the subject biomarkers, as compared to a control, is indicative of an increased risk of PTB. In one embodiment, a decrease in the level of one of the subject biomarkers, as compared to a control, is indicative of an increased risk of PTB.

In one embodiment, when Mobiluncus mulieris is present in the sample, a low SLPI level as compared to a control is indicative of an increased risk of PTB. In one embodiment, a SLPI level in the lowest 25% of a control population is indicative of about 3 fold greater risk of PTB.

In another embodiment, when Mobiluncus mulieris is present in the sample, a low IL-1ra level as compared to a control is indicative of an increased risk of PTB. In one embodiment, an IL-1ra level in the lowest 25% of a control population is indicative of about 2 fold greater risk of PTB.

In yet another embodiment, when Mobiluncus mulieris is present in the sample, a low B-defensin level as compared to a control is indicative of an increased risk of PTB. In one embodiment, a B-defensin level in the lowest 25% of a control population is indicative of about 9 fold greater risk of PTB.

In another embodiment, when Atopobium vaginae is present in the sample, a low IL-1ra level as compared to a control is indicative of an increased risk of PTB. In one embodiment, an IL-1ra level in the lowest 25% of a control population is indicative of about 2 fold greater risk of PTB.

In yet another embodiment, when Atopobium vaginae is present in the sample, a low Beta defensins level as compared to a control is indicative of an increased risk of PTB. In one embodiment, a Beta defensins level in the lowest 25% of a control population is indicative of about 9 fold greater risk of PTB.

### III. Compositions & Kits

The compositions, kits and methods described herein include reagents which are capable of detecting, binding, specifically complexing with, or measuring the level of the subject bacteria. Such reagents include those which are capable of detecting, or measuring the abundance of, said bacteria at the nucleic acid level. Suitable reagents include those for detection by polymerase chain reaction (PCR). Suitable reagents can be purchased commercially, e.g., such as the QIAamp DNA Microbiome kit and the MicroSEQ rDNA PCR kit. In addition, suitable reagents may be designed by the person of skill in the art based on the published sequences of the specific subject bacterium. In one embodiment, the reagents are PCR primers and/or probes. In addition, other suitable components are included to allow for the identification and quantitation of the subject bacteria. Such components include, e.g. enzymes, buffers and deoxynucleotides necessary for reverse transcription and/or PCR, preferably for qualitative and/or quantitative RT-PCR, detectable probes and/or an internal control. The present invention further provides a kit comprising the assay of the invention and optionally instructions for use.

The compositions, kits and methods described herein also, in some embodiments, include reagents which are capable of detecting, binding, specifically complexing with, or measuring the level of expression of the subject biomarkers. Such reagents include those which are capable of detecting, or measuring the level of expression of, said biomarkers at the polypeptide or protein level. In one embodiment, the reagents capable of detecting the biomarker(s) are proteins or polypeptides. In one embodiment, the proteins or polypeptides are antibodies or fragments thereof, e.g., such as those suitable for use in an ELISA.

In one embodiment, at least one reagent is labeled with a detectable label. Suitable labels include, without limitation, an enzyme, a fluorochrome, a luminescent or chemi-luminescent material, or a radioactive material. In another embodiment, at least one reagent is immobilized on a substrate.

In one embodiment, the assay is an enzyme-linked immunosorbent assay (ELISA), and the reagents are thus, appropriate for that format. In another embodiment, the assay is a Meso Scale Discovery (MSD) immunoassay, and the reagents are thus appropriate for that format. In another embodiment, the suitable assay is selected from the group consisting of an immunohistochemical assay, a counter immuno-electrophoresis, a radioimmunoassay, radioimmunoprecipitation assay, a dot blot assay, an inhibition of competition assay, and a sandwich assay. In another embodiment, the assay is one that utilizes electrochemiluminescent detection. In another embodiment, the diagnostic reagent is labeled with a detectable label. In one embodiment, the label is an enzyme, a fluorochrome, a luminescent or chemi-luminescent material, or a radioactive material.

Any combination of the described reagents for the detection of the subject bacteria and/or reagents for the detection of the subject biomarkers can be assembled in a diagnostic kit for the purposes of diagnosing PTB. For example, one embodiment of a diagnostic kit includes reagents for 1, 2, 3, 4, 5, 6, 7, 8, 9, 10, 11, 12, 13, 14, 15, 16, 17, 18, 19, 20, 21 or 22 of the subject bacteria optionally in combination with reagents for 1, 2, 3, 4, 5, 6, 7, 8, 9, 10, 11 or 12 of the subject biomarkers. In one embodiment, one or more of the reagents is associated or bound to a detectable label or bound to a substrate. Still other components of the biomarker signatures, associated with detectable labels or immobilized on substrates provide additional diagnostic kits. Still other components of the biomarker signatures are labeled or immobilized biomarkers or fragments thereof.

For these reagents, the labels may be selected from among many known diagnostic labels, including those described above. Similarly, the substrates for immobilization may be any of the common substrates, glass, plastic, a microarray, a microfluidics card, a chip or a chamber.

It is intended that any of the compositions described herein can be a kit containing multiple reagents or one or more individual reagents. For example, one embodiment of a composition includes a substrate upon which one or more of the reagents are immobilized. In another embodiment, the composition is a kit also contains optional detectable labels, immobilization substrates, optional substrates for enzymatic labels, as well as other laboratory items. In one embodiment, the kit contains a standard for use as a control.

### IV. Methods

In another aspect, methods of predicting the risk of preterm birth in a subject, prior to or during pregnancy, are provided. In one embodiment, the diagnostic method involves correlating the presence or amount of one or more subject bacteria, optionally in combination with the level of one or more subject biomarkers, with a diagnosis of increased risk of PTB. In one embodiment, the diagnostic method involves correlating the level of one or more subject biomarkers, with a diagnosis of increased risk of PTB.

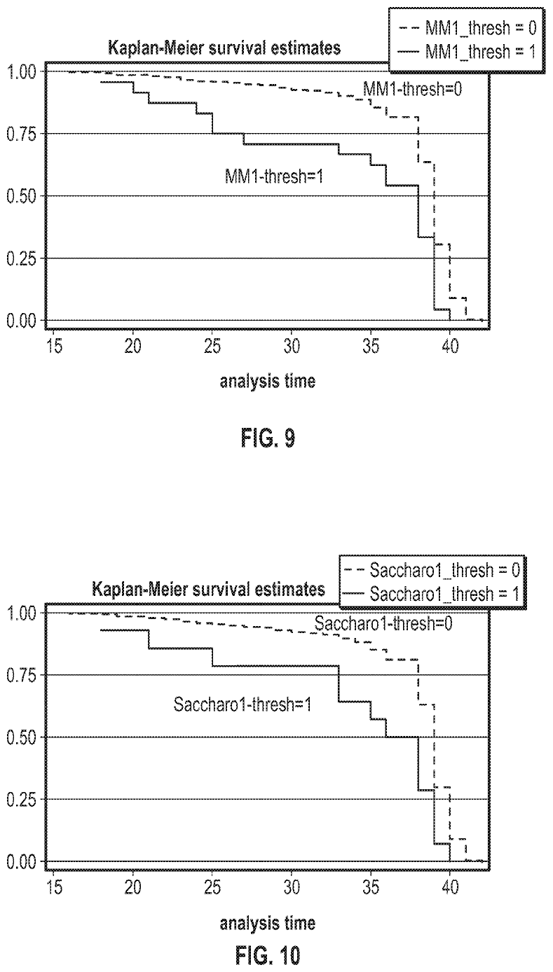

In some embodiments, the presence or absence of a specific bacteria is indicative of an increased risk of PTB. The determination of presence or absence may be made as compared to a control or threshold level. For example, as shown in FIG. 9, the presence of Mobiluncus mulieris above a baseline level (control level), results in increased incidence of PTB. As shown in FIG. 10, the presence of BVAB3 above a baseline level (control level), results in increased incidence of PTB.

In one embodiment, the amount of the one or more subject bacteria and/or the level of the one or more subject biomarkers is compared to one or more control levels to provide the diagnosis. In one embodiment, the predetermined control may be tailored specifically for the sample being tested. For example, for the sample of an African American patient, a predetermined control comprising healthy African American subjects who did not develop PTB may be used. Alternatively, it may be advantageous to use, for example, the mean level of a predetermined control group comprising all subjects with a specific clinical indicator of PTB, i.e., all African American subjects, all smokers, all women with prior PTB, may vary based on the clinical indicator chosen. Alternatively, it may be advantageous to use, for example, the mean level of a predetermined control group comprising all subjects with a specific common characteristic, e.g., race or age. Alternatively, it may be advantageous to use, for example, the mean level of a predetermined control group comprising all healthy subjects with no history of PTB. In another embodiment, the control is an artificial control, such as a standard provided with a kit. In one embodiment, the subject is not yet pregnant.

Similarly, a method for monitoring progression of PTB in a mammalian subject involves correlating the presence or amount of one or more subject bacteria, optionally in combination with the level of one or more subject biomarkers in a biological sample from a mammalian subject having PTB over a given time period. In one embodiment, the method of the invention further comprises repeating the method multiple times during the subject's pregnancy. In one embodiment, the measurement is repeated two times during the pregnancy. In another embodiment, the measurement is repeated three, four, five or more times during the pregnancy. In another embodiment, the subject is being treated for PTL or increased likelihood of PTB and wherein the method enables a determination of the efficacy of the treatment. In one embodiment, the method involves detecting the presence or level of one or more bacterium in a patient sample, the bacterium selected from those of Table 1, optionally in combination with the level of one or more subject biomarkers, in a biological sample from a mammalian subject having PTL over a given time period. The level of the selected bacteria and/or the subject biomarkers, is then compared with the level and/or expression in one or more biological samples of the same subject assayed earlier in time, or before or during treatment. In one embodiment, the comparison can occur by direct comparison with one or more prior assessments of the same patient's status. In another embodiment, the reference may be a negative control comprising subjects with PTB.

In one embodiment, the contacting step comprises forming a direct or indirect complex in the subject's biological sample between a diagnostic reagent for the subject bacteria or subject biomarker and the subject bacteria or subject biomarker in the sample. In yet another embodiment, the contacting step further comprises measuring a level of the complex in a suitable assay. The assay may more require than one assay format. For example, in one embodiment, a PCR based assay is performed when assessing the presence and/or level of the one or more subject bacteria, while an immuno-based assay is performed when assessing the level of expression of the one or more biomarkers. In one embodiment, only one of these assays is done, i.e., only the PCR based assay for the bacteria or only the immunoassay for the biomarkers.

Methods useful in performing the diagnostic steps described herein are known in the art. Such methods include methods based on hybridization analysis of polynucleotides, methods based on sequencing of polynucleotides, proteomics-based methods or immunochemistry techniques. The most commonly used methods known in the art for the quantification of the amount or abundance of bacteria in a sample include, flow cytometry, and PCR-based methods, such as reverse transcription polymerase chain reaction (RT-PCR) or qPCR. In one embodiment, the PCR-based method utilizes 16S rRNA gene identification. See, e.g., Srinivasan et al, Use of 16S rRNA Gene for Identification of a Broad Range of Clinically Relevant Bacterial Pathogens, PLOS One, February 2015, which is incorporated herein by reference.

The methods described herein are not limited by the particular techniques selected to perform them. Exemplary commercial products for generation of reagents or performance of assays include TRI-REAGENT, Qiagen RNeasy mini-columns, MASTERPURE Complete DNA and RNA Purification Kit (EPICENTRE®, Madison, Wis.), Paraffin Block RNA Isolation Kit (Ambion, Inc.) and RNA Stat-60 (Tel-Test), the MassARRAY-based method (Sequenom, Inc., San Diego, Calif.), differential display, amplified fragment length polymorphism (iAFLP), and BeadArray™ technology (Illumina, San Diego, Calif.) using the commercially available Luminex100 LabMAP system and multiple color-coded microspheres (Luminex Corp., Austin, Tex.) and high coverage expression profiling (HiCEP) analysis.

In one embodiment, the immunoassay is an enzyme-linked immunosorbent assay (ELISA). In another embodiment, the suitable assay is selected from the group consisting of an immunohistochemical assay, a counter immuno-electrophoresis, a radioimmunoassay, radioimmunoprecipitation assay, a dot blot assay, an inhibition of competition assay, and a sandwich assay.

In another embodiment, one or more of the diagnostic reagents is labeled with a detectable label. In one embodiment, the label is an enzyme, a fluorochrome, a luminescent or chemi-luminescent material, or a radioactive material. In another embodiment, the diagnostic reagent is an antibody or fragment thereof specific for one of the subject biomarkers.

In yet another embodiment of the method of the invention, the measuring is performed by a computer processor or computer-programmed instrument that generates numerical or graphical data useful in diagnosing the likelihood of PTB. In one embodiment, the method of the invention further comprises coupling the relationship of the sample bacteria and/or biomarker level with the predetermined control level.

As stated above, in one example, the specific diagnostic methodology employed in the method includes detecting the presence or level of bacteria as ribonucleic acid (16S rRNA) and measuring the level of the biomarker(s) as protein (i.e., measuring translation of the protein) using conventional assay technologies. In one embodiment, the presence or level of the one or more subject bacteria is measured in the cervicovaginal fluid the nucleic acid level and the level of the one or more subject biomarkers is measured at either the mRNA or protein levels, respectively by polymerase chain reaction and enzyme-linked immunosorbent assay (ELISA). The specific methodologies that can be employed to perform the diagnostic methods described herein are conventional and may be readily selected and adapted by one of skill in the art.

The measurement of the subject biomarker(s) protein in the biological sample may employ any suitable ligand (reagent), e.g., antibody to detect the protein. Such antibodies may be presently extant in the art or presently used commercially, or may be developed by techniques now common in the field of immunology. As used herein, the term “antibody” refers to an intact immunoglobulin having two light and two heavy chains or any fragments thereof. Thus, a single isolated antibody or fragment may be a polyclonal antibody, a high affinity polyclonal antibody, a monoclonal antibody, a synthetic antibody, a recombinant antibody, a chimeric antibody, a humanized antibody, or a human antibody. The term “antibody fragment” refers to less than an intact antibody structure, including, without limitation, an isolated single antibody chain, a single chain Fv construct, a Fab construct, a light chain variable or complementarity determining region (CDR) sequence, etc. As used herein, the term “antibody” may also refer, where appropriate, to a mixture of different antibodies or antibody fragments that bind to the subject biomarker. Antibodies or fragments useful in the method of this invention may be generated synthetically or recombinantly, using conventional techniques or may be isolated and purified from plasma or further manipulated to increase the binding affinity thereof. It should be understood that any antibody, antibody fragment, or mixture thereof that binds to one of the subject biomarkers as defined above may be employed in the methods of the present invention, regardless of how the antibody or mixture of antibodies was generated.

Similarly, the antibodies may be tagged or labeled with reagents capable of providing a detectable signal, depending upon the assay format employed. Such labels are capable, alone or in concert with other compositions or compounds, of providing a detectable signal. Where more than one antibody is employed in a diagnostic method, e.g., such as in a sandwich ELISA, the labels are desirably interactive to produce a detectable signal. Most desirably, the label is detectable visually, e.g. colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength.

Other label systems that may be utilized in the methods of this invention are detectable by other means, e.g., colored latex microparticles (Bangs Laboratories, Indiana) in which a dye is embedded may be used in place of enzymes to provide a visual signal indicative of the presence of the resulting protein-antibody complex in applicable assays. Still other labels include fluorescent compounds, radioactive compounds or elements. Preferably, an antibody is associated with, or conjugated to a fluorescent detectable fluorochromes, e.g., fluorescein isothiocyanate (FITC), phycoerythrin (PE), allophycocyanin (APC), coriphosphine-O (CPO) or tandem dyes, PE-cyanin-5 (PC5), and PE-Texas Red (ECD). Commonly used fluorochromes include fluorescein isothiocyanate (FITC), phycoerythrin (PE), allophycocyanin (APC), and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC+PE, FITC+PECy5 and PE+PECy7, among others may be used depending upon assay method.

Detectable labels for attachment to antibodies useful in diagnostic assays of this invention may be easily selected from among numerous compositions known and readily available to one skilled in the art of diagnostic assays. The antibodies or fragments useful in this invention are not limited by the particular detectable label or label system employed. Thus, selection and/or generation of suitable antibodies with optional labels for use in this invention is within the skill of the art, provided with this specification, the documents incorporated herein, and the conventional teachings of immunology.

In another aspect, a method of diagnosing and treating a subject for an increased risk of preterm birth is provided. The method includes contacting a sample from the subject with a reagent capable of detecting, binding, specifically complexing with, or measuring the level of one of the bacterium from Table 1. Such reagents are described herein. The subject is diagnosed with an increased risk of preterm birth when the presence, increased relative abundance or increased absolute abundance of a detrimental bacterium is detected. An effective amount of a therapeutic is administered to reduce the risk of PTB. In one embodiment, such therapeutics include agents which make the vaginal space less hospitable to detrimental bacteria. In another embodiment, such therapeutics include agents which make the vaginal space more hospitable to beneficial bacteria. In one embodiment, the therapeutic is a probiotic which is administered to the vaginal space. Such probiotics include those from the genus Lactobacillus. In one embodiment, the probiotic comprises Lactobacillus rhamnosus.

In another embodiment, the therapeutic is vaginal progesterone. In yet another embodiment, the therapeutic is an agent that changes the pH of the vagina.

### V. Examples

The examples that follow do not limit the scope of the embodiments described herein. One skilled in the art will appreciate that modifications can be made in the following examples which are intended to be encompassed by the spirit and scope of the invention.

### Example 1: Distinct Microbiota in the Cervicovaginal Space are Associated with Spontaneous Preterm Birth

Changes in microbial communities have been implicated in both health and disease. Investigations into the association between the cervicovaginal (CV) microbiota and spontaneous preterm birth (sPTB) have been limited in scope and number. This study sought to assess if longitudinal cohort of pregnant women and then to perform validation in a 2nd prospective cohort.

A prospective cohort of singleton pregnancies was enrolled (“M&M”, n=1500). Biospecimens were collected at 3 time points in pregnancy (16-20 (V1), 20-24 (V2), 24-28 (V3) weeks). All cases of PTB were adjudicated by the principal investigator (PI). From the larger cohort, a nested case-control was performed with 80 SPTB cases and 320 term controls that were frequency matched by race to the cases. 16S rRNA gene analyses were performed to characterize the composition and structure of the CV microbiota. The effect of bacteria was quantified as the log ratio between the mean relative abundance at SPTB samples vs. TERM delivery samples. The log ratios were estimated using zero-inflated negative binomial models. A second cohort of woman (“STOP”) with specimens collected between 22-32 weeks was used as validation (N=616).

When performing phylotype analyses, 127 phylotypes were detected in all samples from both cohorts. Significant associations were demonstrated between specific bacteria, in both a positive and negative manner, with sPTB. 37 bacteria were significantly associated with a decreased risk of sPTB while 13 were associated with an increased risk in the primary cohort. Racial differences in these associations were evident (data not shown). The validation cohort confirmed the highly significant associations between specific microbes and SPTB.

Bifidobacterium species were noted to be significantly protective against SPTB at all gestational time points while BVAB2, BVAB3 and g Mobiluncus were associated with a dramatic increase risk of sPTB (all q-values <0.0001).

CV microbiota are significantly associated with sPTB. Targeting the bacteria that are associated with an increased risk of sPTB and/or enhancing the presence of the protective bacteria may serve as new therapies to reduce the rate of PTB.

### Example 2. Integrating Low and High Risk Cervicovaginal Microbiota with Antimicrobial Peptides to Identify Those Women at Greatest Risk for Spontaneous Preterm Birth

Cervicovaginal (CV) microbial communities may interact with specific immune responses that change the cervical epithelial barrier leading to premature cervical remodeling and spontaneous preterm birth (SPTB). We investigated if presence/absence of certain microbiota alters the relationship between antimicrobial peptides (amps) and risk of SPTB.

CV biospecimens were collected in a prospective cohort of singleton pregnancies (“M&M”; n=1500). Using biospecimens at 16-20 weeks, a nested case-control study of 83 cases of confirmed sPTB and 83 race-matched term controls was performed. Four bacteria (Bifidobacterium breve (BB), Bifidobacterium longum (BL), BVAB3, and Mobiluncus mulieris (MM)) were examined with quartiles of four amps (SLPI, IL-1ra, Beta defensins, and Hyaluronidase (HA)). Odds of sPTB were estimated using logistic regression stratified by presence/absence of the bacteria.

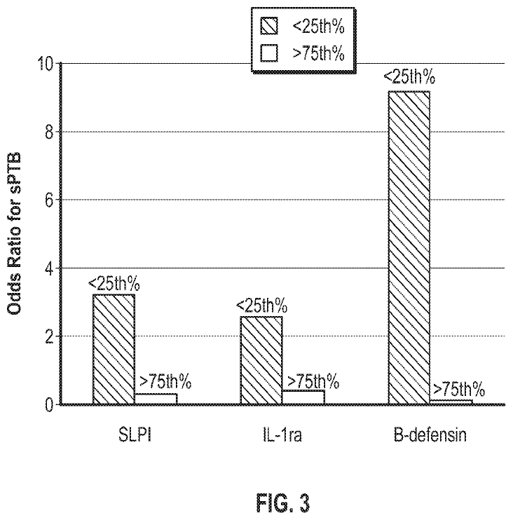

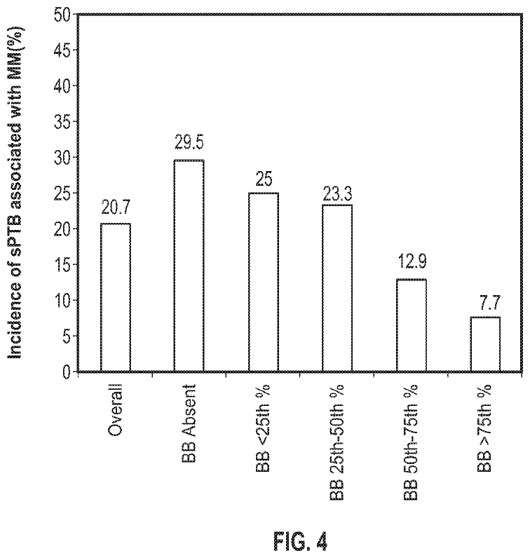

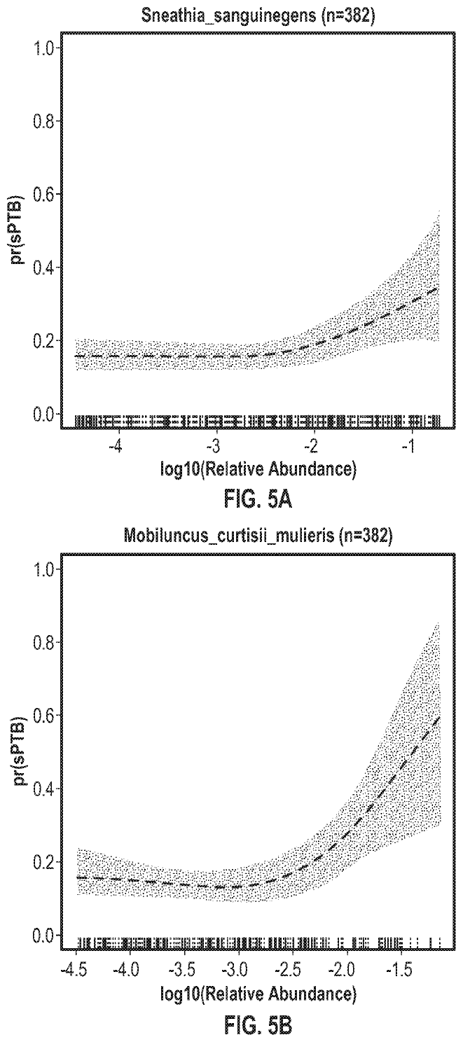

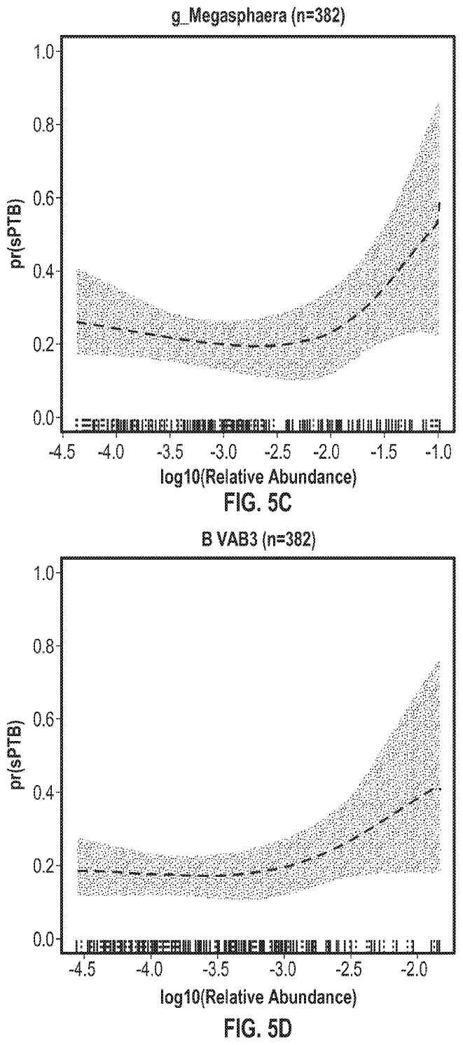

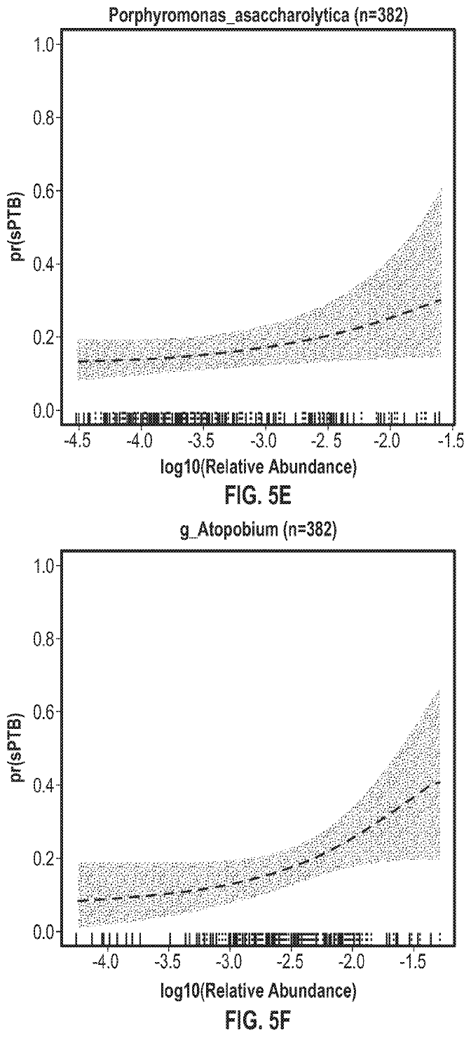

An increased risk of sPTB was found with the lowest quartile (Q1) of SLPI (OR=2.57, 95% CI 1.06-6.25), Beta defensins (OR=4.50, 95% CI 1.79-11.29), and HA (OR=3.85, 95% CI 1.52-9.75) (FIG. 3). Among bacteria, reduced risk of sPTB was seen with BB (OR=0.60, 95% CI 0.36-0.98) (FIG. 4) and BL (OR=0.55, 95% CI 0.34-0.90), and increased risk with MM (OR=1.66, 95% CI 1.02-2.69). There was no association between the biomarkers and bacteria (p>0.05). However, when stratified by presence/absence of bacteria, risk of sPTB for Q1 of Beta defensins doubled with MM and nearly tripled in the absence of BL. In contrast, risk of sPTB with BB and MM and in the absence of BL was halved at Q4 of Beta defensins.

A complex interaction exists between CV microbiota and immune mediators for risk of sPTB. These data suggest that an algorithm incorporating low and high-bacteria with specific mediators of the host immune response may best identify those at risk for sPTB. Once appropriately identified, targeted therapies to reduce sPTB can be studied.

### Example 3: Total Bacteria Load

Total bacterial load (absolute abundance of all bacteria) as a function of gestation age was measured (FIG. 1). There was a significant decrease in the total bacterial load in the second part of gestation in sPTB subjects.

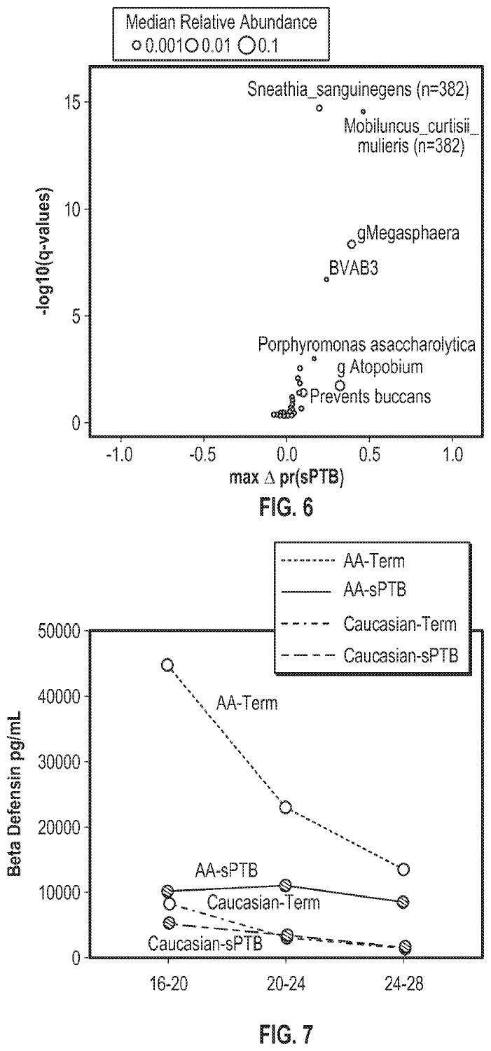

Table 4 below shows the microbes with greatest effect on PTB at 16-20 weeks. See also FIG. 6.

TABLE 4
Maximum change of sPTB risk over all visits with the corresponding p and q-values
for species with significantly non-zero effect size (q-value significance level
0.05) and the amplitude of the effect size of at least 0.1 are shown.

| | gEff | p-val | q-val | n(TERM) | | n(sPTB) | −mode(log₁₀(RA)) |
|---|---|---|---|---|---|---|---|
| Sneathia sanguinegens | 0.1986 | 0 | 0 | 273 | (173) | 64 (46) | 3.885 |
| Mobiluncus curtisii mulieris | 0.4663 | 0 | 0 | 407 | (240) | 84 (56) | 3.954 |
| g Megasphaera | 0.3962 | 1.848e−10 | 4.3434e−09 | 111 | (76) | 38 (28) | 2.94 |
| BVAB3 | 0.2429 | 1.014e−08 | 1.826e−07 | 104 | (76) | 34 (26) | 4.125 |
| Porphyromonas asaccharolytica | 0.1673 | 7.381e−05 | 0.001063 | 193 | (122) | 38 (30) | 3.67 |
| g Atopobium | 0.3251 | 0.002356 | 0.01885 | 126 | (81) | 32 (23) | 2.4 |
| Prevotella buccalis | 0.1042 | 0.006097 | 0.03991 | 211 | (133) | 35 (26) | 3.197 |

### Example 4: Beta Defensin

CV beta defensins levels were measured in African American and Caucasian women throughout pregnancy. FIG. 7 shows that beta defensin levels are decreased in sPTB pregnancies as compared to term counterparts.

### Example 5: Additional Immune Responses

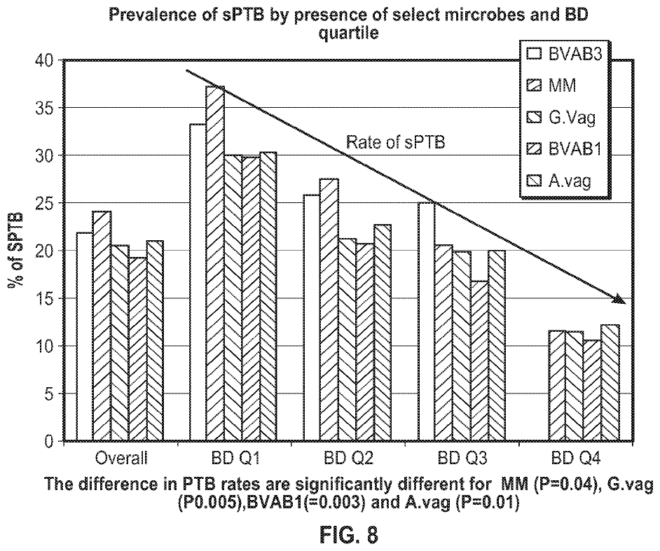

As it is unlikely that one immune mediator will be sufficient to understand the totality and importance of the CV immune response, we investigated the association between the 3 immune mediators studied and sPTB. To perform these analyses, we characterized the immune mediators into quartiles with Q1 being lowest levels and Q4 being highest levels. FIG. 8 shows that increasing quartiles of human beta defensin decreases rate of PTB associated with specific high-risk bacteria.

In women who had Q1 levels for both IL1RA and BD, 77.8% had a sPTB while 22.2% had a term birth. In contrast, in women who had Q4 levels for both BD and IL1RA, 11.1% had a SPTB and 88.9% had a term birth. These data suggest a strong interplay between ‘protective’ immune mediators and the risk of sPTB.

In addition, we examined levels of BD, IL1RA and high-risk CV bacteria. The presence of Mobiluncus mulieris in a woman with Q1 BD and Q1 IL1RA has approximately a 5-fold higher risk of sPTB than the women with Mobiluncus mulieris but with Q4 of both BD and IL1RA.

Even more pronounced effects were seen with Atopobium vaginae. In those with Atopobium vaginae with Q1 BD and Q1 ILRA, there is a 7.5 fold higher risk for sPTB than those with Atopobium vaginae but with Q4 for both BD and IL RA.

### Specific Embodiments

1. A composition comprising at least one reagent capable of detecting, binding, specifically complexing with, or measuring the level of one of a bacterium in a sample selected from:
a. Bifidobacterium breve;
b. Bifidobacterium longum;
c. Prevotella genogroup 4;
d. Mobiluncus mulieris;
e. Arcanobacterium hippocoleae;
f. Streptococcus salivarius;
g. Peptoniphilus indolicus;
h. Gemella;
i. Eubacterium rectale;
j. BVAB2;
k. BVAB3;
l. Lactobacillus rhamnosus;
m. Sneathia sanguinegens;
n. Lactobacillus gasseri;
o. g_Megasphaera (any species in genus);
p. BVAB1;
q. Porphyromonas asaccharolytica;
r. g Atopobium (any species in genus);
s. g_Prevotella (any species in genus, e.g., Prevotella buccalis);
t. Atopobium vaginae;
u. Peptostrepococcus anaerobius;
v. Gardnerella vaginalis;
w. Lactobacillus crispatus; and
x. Lactobacillus iners.
   2. The composition of claim 1, comprising multiple reagents, each capable of detecting, binding, specifically complexing with, or measuring the level of one of a bacterium selected from (a)-(x).
   3. The composition of claim 2, comprising 3, 4, 5, 6, 7, 8, 9, 10, 11, 12, 13, 14, 15, 16, 17, 18, 19, 20, 21 or all 22 of said reagents.
   4. A composition comprising at least one reagent capable of detecting, binding, specifically complexing with, or measuring the level of one of a bacterium in a sample selected from:
a. Sneathia sanguinegens; and
b. Mobiluncus mulieris;
c. g_Megasphaera (any species in genus);
d. BVAB3;
e. Porphyromonas asaccharolytica;
f. g Atopobium (any species in genus); and
g. g_Prevotella (any species in genus, e.g., Prevotella buccalis)
   5. The composition of claim 4, comprising multiple reagents, each capable of detecting, binding, specifically complexing with, or measuring the level of one of a bacterium selected from (a)-(g).
   6. The composition of claim 5, comprising 3, 4, 5, 6, or 7 of said reagents.
   7. The composition according to any of claims 1 to 6, further comprising a reagent capable of detecting, binding, specifically complexing with, or measuring the expression of a biomarker selected from:

i. beta defensins;

ii. SLPI;

iii. IL-1Ra;

iv. MIP1 alpha;

v. MIP1beta;

vi. IL-1b;

vii. IL-6;

viii. MCP-1;

ix. IL-1a;

x. Interferon-gamma; and

xi. Interferon-epsilon.

8. The composition according to any of claims 1 to 6, wherein the reagents capable of detecting the bacteria are PCR reagents.
9. The composition according to claim 7 or claim 8, wherein the reagents capable of detecting the biomarker(s) are proteins or polypeptides.
10. The composition according to claim 9, wherein the proteins or polypeptides are antibodies or fragments thereof.
11. The composition according to any of claims 1 to 10, wherein at least one reagent is labeled with a detectable label.
12. The composition according to claim 11, wherein said label is an enzyme, a fluorochrome, a luminescent or chemi-luminescent material, or a radioactive material.
13. The composition according to any one of claims 1 to 12, wherein at least one reagent is immobilized on a substrate.
14. A method of detecting the likelihood of occurrence of preterm birth, the method comprising detecting the presence or level of one or more bacterium in a patient sample, the bacterium selected from:
a. Bifidobacterium breve;
b. Bifidobacterium longum;
c. Prevotella genogroup 4;
d. Mobiluncus mulieris;
e. Arcanobacterium hippocoleae;
f. Streptococcus salivarius;
g. Peptoniphilus indolicus;
h. Gemella;
i. Eubacterium rectale;
j. BVAB2;
k. BVAB3;
l. Lactobacillus rhamnosus;
m. Sneathia sanguinegens; and
n. Lactobacillus gasseri;
o. g_Megasphaera (any species in genus);
p. BVAB1;
q. Porphyromonas asaccharolytica;
r. g Atopobium (any species in genus);
s. g_Prevotella (any species in genus, e.g., Prevotella buccalis);
t. Atopobium vaginae;
u. Peptostrepococcus anaerobius;
v. Gardnerella vaginalis;
w. Lactobacillus crispatus; and
x. Lactobacillus iners.
   15. A method of detecting the likelihood of occurrence of preterm birth, the method comprising detecting the presence or level of one or more bacterium in a patient sample, the bacterium selected from:
a. Snethia sanguingenous; and
b. Mobiluncus mulieris;
c. g_Megasphaera (any species in genus)
d. BVAB3
e. Porphyromonas asaccharolytica
f. g Atopobium (any species in genus); and
g. g_Prevotella (any species in genus, e.g., Prevotella buccalis)
   16. The method according to claim 14 or claim 15, further comprising measuring the level of one or more of

i. beta defensins;

ii. SLPI;

iii. IL-1Ra;

iv. MIP1 alpha;

v. MIP1beta;

vi. IL-1b;

vii. IL-6;

viii. MCP-1;

ix. IL-1a;

x. Interferon-gamma; and

xi. Interferon-epsilon.

17. The method according to any of claims 14 or 15, comprising contacting the patient sample with reagent capable of detecting, binding, specifically complexing with, or measuring the level of one of the bacterium.
18. The method according to claim 17, wherein the sample is contacted with 3, 4, 5, 6, 7, 8, 9, 10, 11, 12, 13 or 14 of said reagents.
19. The method according to any one of claims 14 to 18, wherein the sample is selected from urine or cervicovaginal fluid.
20. The method according to any one of claims 14 to 19, wherein an increased risk of preterm birth is diagnosed when the absolute abundance of one or more subject bacteria is increased as compared to a control level.
21. The method according to any one of claims 14 to 19, wherein an increased risk of preterm birth is diagnosed when the relative abundance of one or more subject bacteria is increased as compared to a control level.
22. The method according to any one of claims 14 to 19, wherein an increased risk of preterm birth is diagnosed when the presence of one or more subject bacteria is detected in the sample.
23. The method according to any one of claims 14 to 19, wherein a decreased risk of preterm birth is diagnosed when the absolute abundance of one or more beneficial bacteria is increased as compared to a control level.
24. The method according to any one of claims 14 to 19, wherein a decreased risk of preterm birth is diagnosed when the relative abundance of one or more detrimental bacteria is increased as compared to a control level.
25. The method according to any one of claims 14 to 19, wherein a decreased risk of preterm birth is diagnosed when the presence of one or more subject bacteria is detected in the sample.
26. The method according to any one of claims 14 to 25, wherein a decrease in the total bacterial load of the sample is indicative of an increased risk of preterm birth.
27. The method according to any one of claims 14 to 26, wherein an increase in the absolute abundance or relative abundance of Staphylococcus warneri is indicative of a decrease in the risk of preterm birth as compared to a control.
28. The method according to any one of claims 14 to 27, wherein an increase in the absolute abundance or relative abundance of Streptococcus equinus is indicative of a decrease in the risk of preterm birth as compared to a control.
29. The method according to any one of claims 14 to 28, wherein a decrease in the absolute abundance or relative abundance of Lactobacillus rhamnosus is indicative of a decrease in the risk of preterm birth as compared to a control.
30. The method according to any one of claims 14 to 29, wherein an increase in the absolute abundance or relative abundance of BVAB3 is indicative of an increase in the risk of preterm birth as compared to a control.
31. The method according to any one of claims 14 to 30, wherein an increase in the absolute abundance or relative abundance of Mobiluncus mulieris is indicative of an increase in the risk of preterm birth as compared to a control.
32. The method according to any one of claims 14 to 31, wherein an increase in the absolute abundance or relative abundance of Sneathia sanguinegens is indicative of a decrease in the risk of preterm birth as compared to a control.
33. The method according to any one of claims 14 to 32, wherein an increase in the absolute abundance or relative abundance of g_Megasphaera is indicative of a decrease in the risk of preterm birth as compared to a control.
34. The method according to any one of claims 14 to 33, wherein an increase in the absolute abundance or relative abundance of Porphyromonas asacchrolytica is indicative of an increase in the risk of preterm birth as compared to a control.
35. The method according to any one of claims 14 to 34, wherein an increase in the absolute abundance or relative abundance of g_Atopobium is indicative of an increase in the risk of preterm birth as compared to a control.
35. The method according to any one of claims 14 to 35, wherein an increase in the absolute abundance or relative abundance of Prevotella buccalis is indicative of an increase in the risk of preterm birth as compared to a control.
36. The method according to any one of claims 14 to 35, wherein an increase in the absolute abundance or relative abundance of Mobiluncus mulieris and/or BVAB3 is indicative of an about 2 times or greater risk of preterm birth compared to control.
37. The method according to any one of claims 14 to 36, wherein, when Mobiluncus mulieris is present in the sample, a low SLPI level as compared to a control is indicative of an increased risk of PTB.
38. The method according to claim 37, wherein a SLPI level in the lowest 25% of a control population is indicative of about 3 times greater risk of PTB.
39. The method according to any one of claims 14 to 38, wherein, when Mobiluncus mulieris is present in the sample, a low IL-1Ra level as compared to a control is indicative of an increased risk of PTB.
40. The method according to claim 39, wherein a IL-1Ra level in the lowest 25% of a control population is indicative of about 2 times greater risk of PTB.
41. The method according to any one of claims 14 to 40, wherein, when Mobiluncus mulieris is present in the sample, a low Beta defensins level as compared to a control is indicative of an increased risk of PTB.
42. The method according to any one of claims 14 to 41, wherein, when Mobiluncus mulieris is present in the sample, a low Beta defensins level and/or IL1Ra as compared to a control is indicative of an increased risk of PTB.
43. The method according to claim 42, wherein a Beta defensins level and/or IL-1Ra level in the lowest 25% of a control population is indicative of about 5 times greater risk of PTB.
44. The method according to any one of claims 14 to 44, wherein, when A. vaginae is present in the sample, a low B-defensin level and/or IL1Ra as compared to a control is indicative of an increased risk of PTB.
45. The method according to claim 44, wherein a Beta defensins level and/or IL-1Ra level in the lowest 25% of a control population is indicative of about 7 times greater risk of PTB.
46. The method according to claim 41, wherein a Beta defensins level in the lowest 25% of a control population is indicative of about 9 times greater risk of PTB.
47. The method according to any one of claims 14 to 46, wherein, in the presence of Moliluncus mulieris in the sample, the absence of Bifidobacterium breve, or a Bifidobacterium breve level in the lowest 25% of a control population, is indicative of increased risk of PTB.
48. The method according to any one of claims 14 to 46, wherein, in the presence of Moliluncus mulieris in the sample, a Bifidobacterium breve level in the highest 50% or 75% of a control population is indicative of decreased risk of PTB.
49. The method according to any one of claims 14 to 48, wherein the sample is obtained from a subject at the beginning of pregnancy to about 28 weeks of pregnancy.
49. The method according to any one of claims 14 to 48, wherein the sample is obtained from a subject prior to pregnancy.
50. The method according to any one of claims 14 to 41, further comprising treating the subject when an increased risk of PTB is diagnosed.
51. A method of diagnosing and treating a subject for an increased risk of preterm birth, the method comprising:

a. contacting a sample from the subject with a reagent capable of detecting, binding, specifically complexing with, or measuring the level of one of the bacterium from Table 1;

b. diagnosing the subject with an increased risk of preterm birth when the presence, increased relative abundance or increased absolute abundance of a detrimental bacterium is detected;

c. administering an effective amount of a therapeutic to reduce the risk of PTB.

52. The method according to claim 50 or 51, wherein the therapeutic is a probiotic which is administered to the vaginal space.
53. The method according to claim 52, wherein the probiotic comprises a Lactobacillus species.
54. The method according to claim 52, wherein the probiotic comprises Lactobacillus rhamnosus.
55. The method according to claim 50 or 51, wherein the therapeutic is vaginal progesterone.
56. The method according to claim 50 or 51, wherein the therapeutic is an agent which changes the pH of the vagina.
57. The method according to any one of claims 14 to 56, wherein the control is selected from:
(a) a healthy pregnant mammalian subject at the same time of pregnancy as the subject;
(b) a healthy pregnant mammalian subject who did not develop preterm birth;
(c) a population of multiple subjects (a) or (b);
(d) the same subject at an earlier time in the pregnancy; and
(e) the same subject prior to pregnancy.
   58. Use of the composition according to any one of claims 1 to 13, for use in detecting the risk of PTB.
   59. A kit comprising any of the compositions of claim 1 to 13.
   60. A kit comprising:

(a) multiple reagents, each capable of detecting, binding, specifically complexing with or measuring the level of one of the bacterium from Table 1; and

(b) multiple ligands, each capable of detecting, binding, specifically complexing with, or measuring the expression of a biomarker from Table 2.

Each and every patent, patent application, and publication, including websites cited herein, as well as U.S. Provisional Patent Application No. 62/441,862, is hereby incorporated herein by reference in its entirety. While this invention has been disclosed with reference to specific embodiments, it is apparent that other embodiments and variations of this invention are devised by others skilled in the art without departing from the true spirit and scope of the invention. The appended claims include such embodiments and equivalent variations.

1. A composition comprising multiple reagents each capable of detecting, binding, specifically complexing with, or measuring the level of one of a bacterium in a sample selected from:
a. Bifidobacterium breve;
b. Bifidobacterium longum;
c. Prevotella genogroup 4;
d. Mobiluncus mulieris;
e. Arcanobacterium hippocoleae;
f. Streptococcus salivarius;
g. Peptoniphilus indolicus;
h. Gemella;
i. Eubacterium rectale;
j. BVAB2;
k. BVAB3;
l. Lactobacillus rhamnosus;
m. Sneathia sanguinegens; and
n. Lactobacillus gasseri;
o. g_Megasphaera (any species in genus);
p. BVAB1;
q. Porphyromonas asaccharolytica;
r. g Atopobium (any species in genus, e.g., Atopobium vaginae);
s. g_Prevotella (any species in genus, e.g., Prevotella buccalis);
t. Peptostrepococcus anaerobius;
u. Gardnerella vaginalis;
v. Lactobacillus crispatus; and
w. Lactobacillus iners.

2. (canceled)

3. The composition of claim 1, comprising 3, 4, 5, 6, 7, 8, 9, 10, 11, 12, 13, 14, 15, 16, 17, 18, 19, 20, 21, 22, or all 23 of said reagents.

4. The composition of claim 1, comprising multiple reagent each capable of detecting, binding, specifically complexing with, or measuring the level of one of a bacterium in a sample selected from:
m. Sneathia sanguinegens; and
d. Mobiluncus mulieris;
o. g_Megasphaera (any species in genus);
k. BVAB3;
q. Porphyromonas asaccharolytica;
r. g Atopobium (any species in genus); and
s. g_Prevotella (any species in genus, e.g., Prevotella buccalis)

5-6. (canceled)

7. The composition according to claim 1, further comprising a reagent capable of detecting, binding, specifically complexing with, or measuring the expression of a biomarker selected from:
i. beta defensins;
ii. SLPI;
iii. IL-1Ra;
iv. MIP1 alpha;
v. MIP1beta;
vi. IL-1b;
vii. IL-6;
viii. MCP-1;
ix. IL-1a;
x. Interferon-gamma; and
xi. Interferon-epsilon.

8. The composition according to claim 1, wherein the reagents capable of detecting the bacterium are PCR reagents.

9. The composition according to claim 1, wherein the reagents capable of detecting the biomarker(s) are proteins or polypeptides.

10. The composition according to claim 9, wherein the proteins or polypeptides are antibodies or fragments thereof.

11. The composition according to claim 1, wherein at least one reagent is labeled with a detectable label.

12. The composition according to claim 11, wherein said label is an enzyme, a fluorochrome, a luminescent or chemi-luminescent material, or a radioactive material.

13. The composition according to claim 1, wherein at least one reagent is immobilized on a substrate.

14. A method of detecting the likelihood of occurrence of preterm birth, the method comprising detecting the presence or level of one or more bacterium in a patient sample, the bacterium selected from:
a. Bifidobacterium breve;
b. Bifidobacterium longum;
c. Prevotella genogroup 4;
d. Mobiluncus mulieris;
e. Arcanobacterium hippocoleae;
f. Streptococcus salivarius;
g. Peptoniphilus indolicus;
h. Gemella;
i. Eubacterium rectale;
j. BVAB2;
k. BVAB3;
l. Lactobacillus rhamnosus;
m. Sneathia sanguinegens; and
n. Lactobacillus gasseri;
o. g_Megasphaera (any species in genus);
p. BVAB1;
q. Porphyromonas asaccharolytica;
r. g Atopobium (any species in genus);
s. g_Prevotella (any species in genus, e.g., Prevotella buccalis);
t. Atopobium vaginae;
u. Peptostrepococcus anaerobius;
v. Gardnerella vaginalis;
w. Lactobacillus crispatus; and
x. Lactobacillus iners,
and diagnosing the subject with an increased risk of preterm birth when the presence, increased relative abundance or increased absolute abundance of a detrimental bacterium is detected as compared to a control.

15. The method of claim 14, wherein the bacterium are selected from:
m. Sneathia sanguinegens; and
d. Mobiluncus mulieris;
o. g_Megasphaera (any species in genus);
k. BVAB3;
q. Porphyromonas asaccharolytica;
r. g Atopobium (any species in genus); and
s. g_Prevotella (any species in genus, e.g., Prevotella buccalis)

16. The method according to claim 14, further comprising measuring the level of one or more of
i. beta defensins;
ii. SLPI;
iii. IL-1Ra;
iv. MIP1 alpha;
v. MIP1beta;
vi. IL-1b;
vii. IL-6;
viii. MCP-1;
ix. IL-1a;
x. Interferon-gamma; and
xi. Interferon-epsilon.

17. The method according to claim 14, comprising contacting the patient sample with reagent capable of detecting, binding, specifically complexing with, or measuring the level of one of the bacterium.

18. The method according to claim 17, wherein the sample is contacted with 3, 4, 5, 6, 7, 8, 9, 10, 11, 12, 13, 14, 15, 16, 17, 18, 19, 20, 21, 22 or all 23 of said reagents.

19. The method according to claim 14, wherein the sample is selected from urine or cervicovaginal fluid.

20. A method of diagnosing and treating a subject for an increased risk of preterm birth, the method comprising:
i. contacting a sample from the subject with a reagent capable of detecting, binding, specifically complexing with, or measuring the level of one of the bacterium selected from
a. Bifidobacterium breve;
b. Bifidobacterium longum;
c. Prevotella genogroup 4:
d. Mobiluncus mulieris
e. Arcanobacterium hippocoleae;
f. Streptococcus salivarius;
g. Peptoniphilus indolicus;
h. Gemella;
i. Eubacterium rectale;
j. BVAB2;
k. BVAB3
l. Lactobacillus rhamnosus;
m. Sneathia sanguinegens; and
n. Lactobacillus gasseri;
o. g_Megasphaera (any species in genus);
p. BVAB1;
g. Porphyromonas asaccharolytica;
r. g Atopobium (any species in genus);
s. g_Prevotella (any species in genus, e.g., Prevotella buccalis);
t. Atopobium vaginae;
u. Peptostrepococcus anaerobius;
v. Gardnerella vaginalis;
w. Lactobacillus crispatus; and
x. Lactobacillus iners,
ii. diagnosing the subject with an increased risk of preterm birth when the presence, increased relative abundance or increased absolute abundance of a detrimental bacterium is detected as compared to a control;
iii. administering an effective amount of a therapeutic to reduce the risk of PTB.

21. The method according to claim 20, wherein the therapeutic is a probiotic which is administered to the vaginal space.

22. The method according to claim 20, wherein the therapeutic is vaginal progesterone.

23. The method according to claim 20, wherein the therapeutic is an agent which changes the pH of the vagina.

24. The method according to claim 20, wherein the control is selected from:
(a) a healthy pregnant mammalian subject at the same time of pregnancy as the subject;
(b) a healthy pregnant mammalian subject who did not develop preterm birth;
(c) a population of multiple subjects (a) or (b);
(d) the same subject at an earlier time in the pregnancy; and
(e) the same subject prior to pregnancy.

25-27. (canceled)
